(19) United States
(12) Patent Application Publication
(10) Pub. No.: US 2011/0105500 A1
(43) Pub. Date: May 5, 2011

(54) PYRAZINE SUBSTITUTED PURINES

(75) Inventors: Harish Kumar Mysore Nagaraj, Singapore (SG); Meredith Williams, Singapore (SG); Chang Kai Soh, Singapore (SG)

(73) Assignee: S*BIO Pte Ltd., Republic of Singapore (SG)

(21) Appl. No.: 13/001,099

(22) PCT Filed: Jun. 25, 2009
(86) PCT No.: PCT/SG09/00233

§ 371 Date: Jan. 10, 2011

Related U.S. Application Data
(60) Provisional application No. 61/076,297, filed on Jun. 27, 2008.
(60) Provisional application No. 61/119,746, filed on Dec. 4, 2008.

(51) Int. Cl. A61K 31/5355; C07D 413/14; C12N 9/99; A61P 35/00

(52) U.S. Cl. 514/234.2; 544/118; 435/184

(57) ABSTRACT

The present invention relates to purine compounds of formula (I) that are useful as kinase inhibitors. More particularly, the present invention relates to purine compounds, methods for their preparation, pharmaceutical compositions containing these compounds and uses of these compounds in the treatment of proliferative conditions or disorders. These compounds may be useful as medicaments for the treatment of a number of proliferative conditions or disorders including tumours and cancers as well as other disorders or conditions related to or associated with PI3K and/or mTOR kinases.

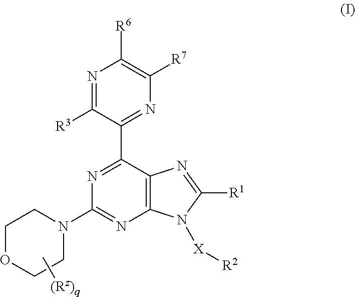

### FIELD

The invention relates to purine compounds that may be useful as kinase inhibitors. More particularly, the invention relates to 2-(morpholin-4-yl), 6-(pyrazin-2-yl) substituted purine derivatives, methods for their preparation, pharmaceutical compositions containing these compounds and uses of these compounds in the treatment of certain medical conditions such as kinase related disorders/conditions.

### BACKGROUND

The search for kinase inhibitors has proven to be a fruitful area for the development of useful pharmaceutically active substances. Kinases, which are alternatively known as phosphotransferases, are enzymes that transfer phosphate groups from high energy donor molecules (for example ATP) to specific target molecules (typically called substrates) in a process termed phosphorylation. One of the largest groups of kinases is the protein kinases which act on and modify the activity of specific proteins. As a result of this activity these kinases are involved in a number of cellular processes such as in signalling and to prime the cell for biochemical reactions in metabolism. Certain cellular signalling processes have been implicated as important in a number of medical conditions and the effective inhibition of certain cell signalling processes therefore provides the potential to stop these conditions developing. Accordingly, kinases represent an attractive target for medicinal chemists as the provision of kinase inhibitors potentially allows for certain signalling processes to be controlled leading to the control of certain medical conditions.

One family of kinases associated with undesirable medical conditions in the body are the phosphoinositide 3-kinase (PI3) family of kinases which are involved in a wide range of cellular events such as cell migration, cell proliferation, oncogenic transformation, cell survival, signal transduction and intracellular trafficking of proteins. This family of kinases has recently been the focus of much research aimed at developing therapies for a range of indications.

The phosphoinositide 3-kinase (PI3) family is a group of enzymes that generate phosphatidylinositol ‘second messengers’. These lipids are subsequently involved in a wide range of physiological processes. In mammalian cells, the large PI3K family has been categorized into three classes, referred to as class I, class II, and class III respectively, each of which has its own characteristics in terms of molecular structure and substrate specificity. For example the preferred in vivo substrate of the class I PI3K is phosphatidylinositol-4,5 bisphosphate, which is phosphorylated to yield phosphatidylinositol-3,4,5 trisphosphate. The class I PI3K are further subdivided into Class IA and IB PI3Ks. Class IA enzymes consist of any one of the ‘catalytic’ subunits (p110α, p110β, or p110δ) complexed with any one of the ‘regulatory’ subunits (p85α, p85β or p55γ). Only one Class IB PI3K enzyme exists, and is made up of the p110γ catalytic and the p101 regulatory subunit. There are also three Class II PI3Ks (CIIα, CIIβ, and CIIα) and one Class III PI3K (Vps34).

The class I PI3Ks are the best understood members of this family and are key players of multiple intracellular signalling networks that integrate a variety of signals initiated by many growth factors. The Class IA enzymes are activated by tyrosine kinases (e.g. growth factor receptors), antigen receptors, and cytokine receptors, whilst the Class IB enzyme is activated by ‘G Protein Coupled Receptors’ (GPCRs). In response to activation, the PI3Ks generate lipid second messengers, which bind to, and activate, specific proteins in distinct signal transduction pathways. The signal transduction pathways remain active until phosphatase enzymes, in particular the oncogene PTEN, dephosphorylate the PI3K lipid second messengers.

The PI3K signalling pathway is crucial to many aspects of cell growth and survival via its regulation of widely divergent physiological processes that include cell cycle progression, differentiation, transcription, translation and apoptosis. Constitutive activation of the PI3K pathway has been implicated in both the pathogenesis and progression of a large variety of cancers and there is now a rapidly accumulating body of evidence that demonstrates conclusively that PI3K signalling is frequently deregulated in cancer. The deregulation of PI3K signalling is thought to occur in two different ways. The first is an increase in PI3K signalling resulting from activating gene mutations, amplification and over expression of PI3Ks or upstream receptors that activate PI3Ks. For example, the PI3Kα catalytic subunit is amplified and over expressed in ovarian and cervical cancers. Similarly, upstream receptor tyrosine kinases that activate PI3K are commonly mutated, amplified and over expressed, e.g., EGFR in breast, ovarian and lung cancer.

In addition, activation of the effectors downstream of PI3K can also contribute to deregulation of the PI3K pathway, e.g., Akt/PKB (Protein Kinase B) is over expressed and activated in breast, pancreatic and ovarian cancers among others. Also, the Ras family members, which are involved in PI3K activation, are frequently mutated, e.g. in colorectal and pancreatic cancer. The second mechanism of PI3K deregulation involves loss of the tumor suppressor phosphatase PTEN, which occurs in many aggressive brain tumors, endometrial and breast cancers, and melanomas.

One specific cell signalling pathway mediated by the PI3 family of kinases is the phosphatidylinositol 3-kinase (PI3K)/Akt pathway. This pathway is critically involved in the mediation of cell survival and is a major signalling component downstream of growth factor receptor tyrosine kinases (RTKs). Growth factor RTKs engage the class-Ia PI3K, which is a heterodimer comprised of the p85 regulatory and p110 catalytic subunits. The small GTPase Ras can also recruit and activate PI3K through direct binding to p110. At the cell membrane, PI3K catalyzes the production of the lipid second messenger phosphatidylinositol-3,4,5-triphosphate (PIP3). Subsequently, PIP3 recruits other downstream molecules—particularly the serine-threonine kinases Akt and PDK1—via binding to their pleckstrin-homology (PH) domains. At the membrane, Akt is partially activated through phosphorylation at threonine 308 in its activation loop by PDK1. Additional phosphorylation at serine 473 in the C terminus of Akt results in its full activation. Akt in turn regulates a wide range of target proteins, one of which is mTOR. The levels of PIP3 in the cell are strictly regulated and several lipid phosphatases act to rapidly remove it. Of particular interest is the phosphatase PTEN, which converts PIP3 back to PIP2 and thus shuts off PI3K signalling. The PI3K-Akt signalling pathway regulates many normal cellular processes including cell proliferation, survival, growth, and motility—processes that are critical for tumorigenesis.

The role of the PI3K/Akt pathway in oncogenesis has also been extensively investigated and mutations or altered expression of most of the pathway's components have been widely implicated in many cancers. Gene amplification of p110 occurs in some cases of human ovarian cancer, and amplification of Akt is found in ovarian, breast, and colon cancer. In addition, activating mutations in p85 have been identified in ovarian and colon cancer. Most importantly PTEN has been identified as a major tumor suppressor in humans and loss-of-function mutations in the PTEN gene are extremely common among sporadic glioblastomas, melanomas, prostate cancers, and endometrial carcinomas, and a significant percentage of breast tumors, lung cancers, and lymphomas also bear PTEN mutations. Thus, through a variety of mechanisms, a high percentage of human cancers possess activated PI3K signalling. Significantly, it has been shown that mTOR is important for the oncogenic transformation induced by PI3K and Akt.

In addition to the compelling correlative data presented above, direct proof of the involvement of deregulated PI3K signalling in cancer comes from mouse genetic models. For example, mice with a constitutively activated p85 regulatory subunit of PI3K progress to malignant lymphoma when crossed with p53-knockout mice. Further, retroviral introduction of Akt and Ras caused glioblastomas in mice. Taken together, all these data provide strong validation for the development of novel anticancer strategies targeted at PI3Ks. Indeed recent interest in PI3K inhibitors has been intense with a number of compounds now in development having demonstrated anti-tumor activity in animal models. The most advanced compounds are now undergoing evaluation in phase I clinical trials. Accordingly compounds that are PI3K inhibitors would be expected to show interesting biological activity as PI3K inhibitors have the potential to block the PI3K/Akt signalling pathway and thereby form the basis of therapy in disease involving deregulation of this pathway.

Another area that has received attention has been the serine/threonine kinases. One serine/threonine kinase that has attracted significant interest is the mammalian target of rapamycin (commonly abbreviated as mTOR).

mTOR is a serine/threonine kinase of 289 kDa and is a PI3K-like kinase that links mitogenic stimuli and nutrient status to cell growth and division. mTOR was discovered during studies conducted to understand the mechanism of action of rapamycin. Upon entering cells, rapamycin binds to its intracellular target FKBP12 and the complex then binds to and specifically inhibits mTOR. mTOR was, therefore, also named FKBP-RAP associated protein (FRAP), RAP FKBP12 target (RAFT1) and RAP target (RAPT1). Cells responsible for organ rejection stop growing due to rapamycin's ability to inhibit the anabolic signals coordinated by mTOR. Since inhibition of cell growth represents a valid target for treating cancer, designing new drugs that inhibit mTOR will potentially have therapeutic value.

In humans, mTOR mediates anabolic signals from 2 sources namely nutrients that pass into the cell and activated growth factor receptors. It exists in at least two distinct complexes: a rapamycin-sensitive complex, referred to as mTOR complex 1 (mTORC1), defined by its interaction with the accessory protein raptor (regulatory-associated protein of mTOR). The normal activation of mTOR results in an increase in protein translation because mTORC1 phosphorylates and activates the translation regulators eukaryotic initiation factor 4E-binding protein 1 and ribosomal p70 S6 kinase. Therefore, by inhibiting mTOR, rapamycin causes a decrease in phosphorylation of these effectors, and a decrease in protein synthesis, effectively blocking the pro-growth actions of mTOR.

The second complex, mTOR complex 2 (mTORC2), is rapamycin-insensitive and is defined by its interaction with rictor (rapamycin-insensitive companion of mTOR). mTORC2 is involved in the regulation of the pro-survival kinase Akt/PKB by phosphorylating it on S473. Together with the phosphorylation of T308 by PDK1, S473 phosphorylation is necessary for full Akt activation. Recent reports indicate that prolonged treatment with rapamycin in some cells also suppresses the assembly and function of mTORC2 to inhibit Akt and that this property of rapamycin contributes to the anti-apoptotic effects of the drug. mTOR is also one of the main downstream effectors in the phosphatidylinositol 3-kinase (PI3K)/Akt pathway and therefore inhibition of mTOR provides a further opportunity to inhibit, at least in part, the PI3K/Akt pathway.

An additional pathway influenced by mTOR that appears to be particularly important in renal cell carcinoma involves the hypoxia-inducible factor (HIF). With loss of Von Hippel-Lindau (VHL) gene function commonly seen in clear cell renal cell cancer, there is accumulation of the oxygen-sensitive transcription factors HIF-1 and HIF-2. An accumulation of these factors yields increased stimulation of vascular endothelial growth factor (VEGF), platelet-derived growth factor, and transforming growth factor. This effect is augmented by the activation of mTOR, which stimulates both a protein stabilization function and a protein translational function and, thus, increases HIF-1 activity.

It has also been determined that tuberous sclerosis complex gene products, TSC1 and TSC2, function together to inhibit mTOR-mediated downstream signalling. Mutations of these genes occur in tuberous sclerosis and their loss of function yields yet another pathway, which leads to increased activity of mTOR and induces VEGF production. TSC2 also regulates HIF. Thus, studies evaluating the impact of TSC1 and TSC2 mutations demonstrate the connection of increased VEGF and activated is mTOR pathways to angiogenesis.

So far, four mTOR inhibitors have been tested in clinical trials: the prototype rapamycin and three rapamycin derivatives, CCI-779 (temsirolimus), RAD001 (everolimus) and AP23573. Rapamycin, also named sirolimus, is a natural antibiotic produced by Streptomyces hygroscopicus. It was developed initially as an anti-fungal drug directed against Candida albicans, Cryptococcus neoformans, and Aspergillus fumigatus. Later, rapamycin was developed as an immunosuppressive agent and those studies helped in understanding the mechanism of action of this agent. As an anti-cancer agent, rapamycin was shown to inhibit the growth of several murine and human cancer cell lines in a concentration-dependent manner, both in tissue culture and xenograft models. In the sixty tumor cell lines screened at the National Cancer Institute in the USA, general sensitivity to the drug was seen at doses under 2000 ng/ml, more evident in leukemia, ovarian, breast, central nervous system and small cell lung cancer cell lines. In addition, rapamycin inhibits the oncogenic transformation of human cells induced by either PI3K or Akt and has shown metastatic tumor growth inhibition and anti-angiogenic effects in in vivo mouse models.

Based on these pre-clinical results, clinical trials with rapamycin as an anticancer drug were carried out and rapamycin analogues with more favourable pharmaceutical properties were developed. CCI-779, a more water-soluble ester derivative of rapamycin was identified by investigators at Wyeth Ayerst as a non-cytotoxic agent that inhibited tumor cell proliferation. At several non-toxic doses, CCI-779 demonstrated anti-tumor activity alone or in combination with cytotoxic agents in a variety of human cancer models such as gliomas, rhabdomyosarcoma, primitive neuroectodermal tumor such as medulloblastoma, head and neck, prostate, pancreatic and breast cancer cells. Treatment of mice with CCI-779 inhibits P70S6K activity and reduces neoplastic proliferation. As with rapamycin, PTEN-deficient human tumors are more sensitive to CCI-779-mediated growth inhibition than PTEN expressing cells. Specifically, studies in vitro in a panel of eight human breast cancer cell lines showed that six of eight cancer lines studied were inhibited by CCI-779 with IC50 in the low nanomolar range. Two lines, however, were found to be resistant with IC50>1 μM. The sensitive cell lines were estrogen receptor positive or over-expressed HER-2/Neu, or had lost the tumor suppressor gene product PTEN. The main toxicities of CCI-779 included dermatological toxicities and mild myelosuppression (mainly thrombocytemia).

RAD001, 40-O-(2-hydroxyethyl)-rapamycin, is another analogue of rapamycin that can be administrated orally. Its anti-neoplastic activity has been evaluated in different human cancer cell lines in vitro and in xenograft models in vivo with IC50 ranging from 5 to 1800 nM. p70S6K inhibition and anti-neoplastic effects have been shown in these models, with an optimal effect being achieved with 2.5 mg/kg/day in melanoma, lung, pancreas and colon carcinoma. Similarly, RAD001 demonstrated a concentration-dependent anti-tumor activity in a syngenic rat pancreas carcinoma model with an intermittent dosing schedule. RAD001 has also shown anti-angiogenic activity and inhibits human vascular endothelial cell (HUVEC) proliferation. The toxicity reported for RAD001 includes hypercholesterolemia, hypertriglyceridemia, mild leukocytopenia and thrombocytopenia. In a phase I trial performed in patients with advanced cancer, RAD001 displayed a good safety profile with mild to moderate skin and mucous toxicity up to 30 mg weekly. Preliminary efficacy results showed an objective response in a patient with non-small cell lung carcinoma.

AP23573 is the latest rapamycin analog to be reported in clinical development. It is a phosphorus-containing compound synthesized with the aid of computational modelling studies. AP23573 was found to be stable in organic solvents, aqueous solutions at a variety of pHs and in plasma and whole blood, both in vitro and in vivo and has shown potent inhibition of diverse human tumor cell lines in vitro and as xenografts implanted into nude mice, alone or in combination with cytotoxic or targeted agents. In phase I trials, AP23573 was administered intravenously daily for 5 days every 2 weeks. Dose-limiting toxicity is severe grade 3 oral mucositis occurring during the first cycle. Other side effects seem to be moderate, including minor to moderate episodes of mucositis, fatigue, nausea, rash, anaemia, neutropenia, diarrhoea, hyperlipidemias and thrombocytopenia. Preliminary anti-tumor activity is observed at all dose levels.

There is thus a plethora of studies that demonstrate that mTOR inhibitors can improve cancer patient survival. However, rapamycin and its analogues have not shown universal anti-tumor activity in early clinical trials. Response rates vary among cancer types from a low of less than 10% in patients with glioblastomas and advanced renal-cell cancer to a high of around 40% in patients with mantle-cell lymphoma. Knowledge of the status of PTEN and PI3K/Akt/mTOR-linked pathways might help in the selection of tumor types that will respond to mTOR inhibitors. Furthermore, because many tumor types still do not respond to single agent therapy with rapamycin derivatives, it is important to continue the search for factors predictive of resistance or sensitivity to mTOR inhibitors. Of particular interest will be molecules that directly inhibit mTOR kinase activity, the assumption being that such molecules will inhibit both mTORC1 and mTORC2. Such an inhibitor might be beneficial for treating tumors with elevated Akt phosphorylation and might down-regulate the growth, proliferation and survival effects that are associated with Akt activation. If mTOR-rictor is a crucial activator of Akt-dependent survival processes, such a drug might promote apoptosis in tumor cells that have adapted to Akt-dependent regulatory mechanisms.

Accordingly, mTOR and PI3 have been identified as protein kinases that are involved in a number of disorders, and compounds that target one or more of these kinases should display useful biological activity. Accordingly, compounds that are mTOR and/or PI3K inhibitors have the potential to provide further biologically active compounds that would be expected to have useful, improved pharmaceutical properties in the treatment of kinase related conditions or disorders such as cancer and other proliferative disorders.

In addition compounds that inhibit both mTOR and PI3K simultaneously may be expected to provide powerful antitumor activity as these compounds would be expected to be active on both a number of pathways and on multiple points in the PI3K/Akt/mTOR pathway. A number of dual inhibitors of this type are now being investigated in a clinical setting for the first time (e.g. BEZ235, XL765, GDC0941, PX866, SF1126) and there is therefore the need to develop further compounds of this type.

### SUMMARY

The invention provides compounds of formula (I):

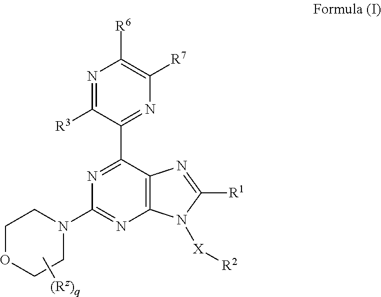

wherein:

R¹ is selected from the group consisting of: H, halogen and optionally substituted C₁-C₆alkyl;

R² is selected from the group consisting of H, halogen, OH, NO₂, CN, NH₂, optionally substituted C₁-C₁₂alkyl, optionally substituted C₂-C₁₂alkenyl, optionally substituted C₂-C₁₂alkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally substituted C₁-C₁₂alkyloxy, optionally substituted C₂-C₁₂alkenyloxy, optionally substituted C₂-C₁₂alkynyloxy, optionally substituted C₂-C₁₂heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂heterocycloalkenyloxy, optionally substituted C₆-C₁₈aryloxy, optionally substituted C₁-C₁heteroaryloxy, optionally substituted C₁-C₁₂alkylamino, SR⁸, SO₃H, SO₂NR⁸R⁹, SO₂R⁸, SONR⁸R⁹, SOR⁸, COR⁸, COOH, COOR⁸, CONR⁸R⁹, NR⁸COR⁹, NR⁸COOR⁹, NR⁸SO₂R⁹, NR⁸CONR⁸R⁹, NR⁸R⁹, and acyl;

R³ is selected from the group consisting of H, F, Cl, Br, OH, optionally substituted C₁-C₆alkyl, OR⁸, OCOR⁸, CH₂OH, NH₂, NR⁸R⁹, NR⁸COR⁹, and NR⁸SO₂R⁹;

R⁶ is selected from the group consisting of H, OH, OR⁸, OP_{g}^{O}, OCOR⁸, CH₂OH, NH₂, NR⁸R⁹, NR⁸P_{g}^{N}, N(P_{g}^{N})₂, NR⁸COR⁹, and NR⁸SO₂R⁹;

R⁷ is selected from the group consisting of H, F, Cl, Br, OH, OR⁸, OCOR⁸, CH₂OH, NH₂, NR⁸R⁹, NR⁸COR⁹, and NR⁸SO₂R⁹;

each R⁸ and R⁹ is independently selected from the group consisting of H, optionally substituted C₁-C₁₂alkyl, optionally substituted C₂-C₁₂alkenyl, optionally substituted C₂-C₁₂alkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, and optionally substituted C₁-C₁₈heteroaryl, or

R⁸ and R⁹ when taken together with the atoms to which they are attached form an optionally substituted cyclic moiety;

P_{g}^{O} is a protecting group for oxygen;

each P_{g}^{N} is independently a protecting group for nitrogen;

each R^{z} is independently selected from the group consisting of C₁-C₆alkyl, halo-C₁-C₆alkyl, hydroxyC₁-C₆alkyl, C₁-C₆alkyloxyC₁-C₆alkyl, cyanoC₁-C₆alkyl, aminoC₁-C₆alkyl, C₁-C₆alkylaminoC₁-C₆alkyl, and di(C₁-C₆alkyl)aminoC₁-C₆alkyl;

q is an integer selected from the group consisting of 0, 1, 2, 3, and 4.

X is a group of formula (CR¹⁰₂)ₘ;

each R¹⁰ is independently selected from the group consisting of: H and optionally substituted C₁-C₆alkyl;

m is an integer selected from the group consisting of 0, 1, 2, 3 and 4;

or a pharmaceutically acceptable salt, N-oxide, or prodrug thereof.

As with any group of structurally related compounds which possess a particular utility, certain embodiments of variables of the compounds of the formula (I), are particularly useful in their end use application.

In various embodiments q is an integer selected from the group consisting of 0, 1, 2, 3, and 4. In some embodiments q is 4. In some embodiments q is 3. In some embodiments q is 2. In some embodiments q is 1. In some embodiments q is 0.

In some embodiments wherein q is other than 0, each R^{z} may be selected from the group consisting of F, Cl, Br, methyl, trifluoromethyl, and ethyl. The R^{z} substituent may be attached at the 2, 3, 5 or 6 position of the morpholine ring and in circumstances where there are multiple R^{z} substituents each R^{z} substituent is located independently of the others. In some embodiments q is 1 and the R^{z} substituent is located at the 3 position of the morpholine ring. This provides compounds of formula (Ia).

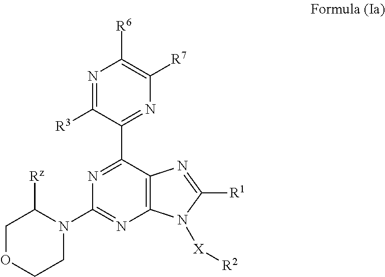

or a pharmaceutically acceptable salt or prodrug thereof,

wherein R¹, R², R³, R⁶, R⁷, R^{z} and X are as defined above.

In some embodiments q is 0. This provides compounds of formula (Ib).

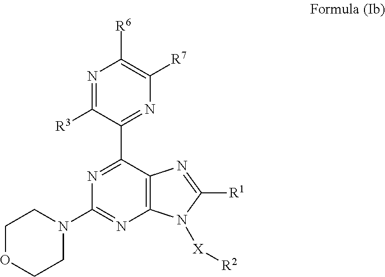

or a pharmaceutically acceptable salt or prodrug thereof,

wherein R¹, R², R³, R⁶, R⁷ and X are as defined above.

In some embodiments R³ is selected from the group consisting of H, OR⁸, and optionally substituted C₁-C₆alkyl.

In some embodiments R³ is OR⁸ where R⁸ is optionally substituted C₁-C₆alkyl. Examples of R³ groups of this type include methoxy, trifluoro-methoxy, ethoxy, isopropoxy, propoxy, and butoxy. In some embodiments R³ is methoxy.

In some embodiments R³ is optionally substituted C₁-C₆alkyl. Examples of R³ groups of this type include methyl, trifluoro-methyl, ethyl, propyl, isopropyl, and butyl. In some embodiments R³ is methyl.

In some embodiments R³ is selected from the group consisting of H, methoxy and methyl. In some embodiments R³ is H.

In some embodiments R⁷ is selected from the group consisting of H, F, Cl, Br, OH and NH₂. In some embodiments R⁷ is H.

In some embodiments R³ and R⁷ are both H.

In some embodiments q=0, R³ is H and R⁷ is H. This provides compounds of formula (Ic):

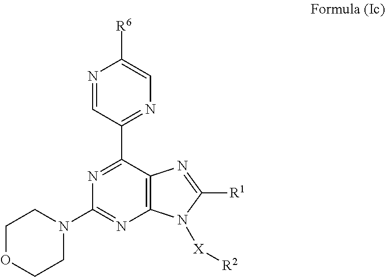

or a pharmaceutically acceptable salt or prodrug thereof,

wherein R¹, R², R⁶, and X are as defined above.

In some embodiments of compounds containing the group R⁸, R⁸ is selected from H and C₁-C₆alkyl. In some embodiments R⁸ is methyl. In some embodiments R⁸ is H.

In some embodiments of the compounds containing the group R⁹, R⁹ is selected from H and C₁-C₆alkyl. In some embodiments R⁹ is methyl. In some embodiments R⁹ is H.

As stated previously X is a group of formula (CR¹⁰₂)ₘ. In some embodiments m is selected from the group consisting of 0, 1, and 2. In some embodiments m is 0 or 1. In some embodiments m is 0. In some embodiments m is 1.

In some embodiments q=0, R³ is H, R⁷ is H and m is o. This provides compounds of formula (II):

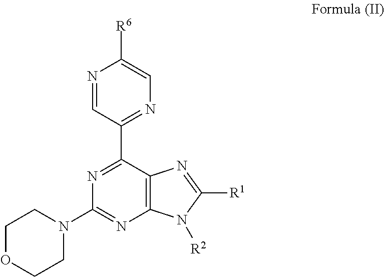

or a pharmaceutically acceptable salt or prodrug thereof,

wherein R¹, R², and R⁶, are as defined above.

In some embodiments q=0, R³ is H, R⁷ is H and m is 1. This provides compounds of formula (III):

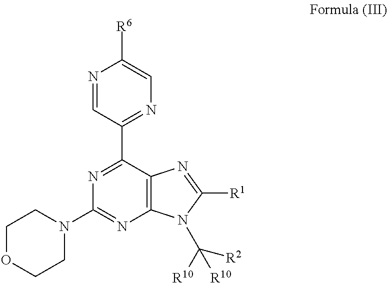

or a pharmaceutically acceptable salt or prodrug thereof,

wherein R¹, R², R⁶ and R¹⁰ are as defined above.

In some embodiments of the compounds of formula (I), (Ia), (Ib), (Ic) and (III) each R¹⁰ is H. In some embodiments each R¹⁰ is independently an optionally substituted C₁-C₆alkyl. In some embodiments one R¹⁰ is H and the other R¹⁰ is H or optionally substituted C₁-C₆alkyl. In some embodiments one R¹⁰ is H and the other is CH₃.

In some embodiments of the compounds of formula (I), (Ia), (Ib), (Ic) and (III) m is 1, one R¹⁰ is H and X is therefore a group of the formula:

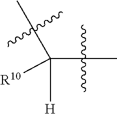

In some embodiments R³ and R⁷ are H, m is 1, q is 0 and one R¹⁰ is H. This provides compounds of the formula (IV):

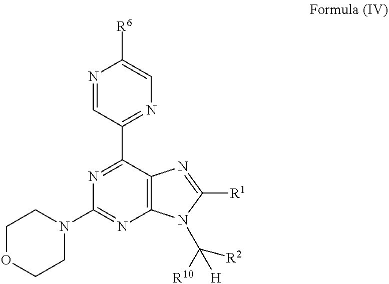

or a pharmaceutically acceptable salt or prodrug thereof,

wherein R¹, R², R⁶ and R¹⁰ are as defined above.

In some embodiments R¹ is selected from the group consisting of H, fluoro, chloro, bromo, methyl, ethyl, isopropyl, propyl, 2-ethyl-propyl, 3,3-dimethyl-propyl, butyl, isobutyl, 3,3-dimethyl-butyl, 2-ethyl-butyl, pentyl, 2-methyl, pentyl, and hexyl. In some embodiments R¹ is H.

In some embodiments R⁶ is selected from the group consisting of H, NH₂ and NR⁸R⁹ wherein R⁸ and R⁹ are as defined above. In some embodiments R⁶ is NH₂.

In some embodiments R² is selected from the group consisting of H, cyano, optionally substituted C₁-C₁₂alkyl, optionally substituted C₂-C₁₂alkenyl, optionally substituted C₂-C₁₂heteroalkyl, optionally substituted C₃-C₁₂cycloalkyl, optionally substituted C₂-C₁₂heterocycloalkyl, optionally substituted C₆-C₁₈aryl, and optionally substituted C₁-C₁₈heteroaryl.

In some embodiments R² is an optionally substituted C₆-C₁₈aryl. In some embodiments of R² the optionally substituted C₆-C₁₈aryl (and hence R²) is a group of the formula:

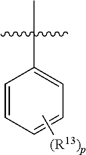

wherein p is an integer selected from the group consisting of 0, 1, 2, 3, 4, and 5;

each R¹³ is independently selected from the group consisting of H, halogen, OH, NO₂, CN, NH₂, optionally substituted C₁-C₁₂alkyl, optionally substituted C₂-C₁₂ alkenyl, optionally substituted C₂-C₁₂alkynyl, optionally substituted C₂-C₁₂heteroalkyl, optionally substituted C₃-C₁₂cycloalkyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally substituted C₁-C₁₂alkyloxy, optionally substituted C₂-C₁₂alkenyloxy, optionally substituted C₂-C₁₂alkynyloxy, optionally substituted C₂-C₁₂heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂heterocycloalkenyloxy, optionally substituted C₆-C₁₈ aryloxy, optionally substituted C₁-C₁₈heteroaryloxy, optionally substituted C₁-C₁₂ alkylamino, SR⁸, SO₃H, SO₂NH₂, SO₂R⁸, SONH₂, SOR⁸, COR⁸, COOH, COOR⁸, CONR⁸R⁹, NR⁸COR⁹, NR⁸COOR⁹, NR⁸SO₂R⁹, NR⁸CONR⁸R⁹, NR⁸R⁹, and acyl, or

any two adjacent R¹³ may, when taken together with each other and the carbon atoms to which they are attached form a cyclic moiety;

where R⁸ and R⁹ are as defined above.

The phenyl group may be unsubstituted or may be optionally substituted with one or more suitable substituent groups. If the phenyl group is substituted then there may be 1, 2, 3, 4 or 5 substituent groups. In some embodiments p is 0, 1 or 2. In some embodiments p is 1. In some embodiments p is 2.

In some embodiments of R² the optionally substituted C₆-C₁₈aryl (and hence R²) is a group of the formula:

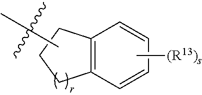

wherein R¹³ is as defined above;

s is an integer selected from the group consisting of 0, 1, 2, 3 and 4;

r is an integer selected from the group consisting of 1, 2, and 3.

In some embodiments r is 1 and the optionally substituted C₆-C₁₈aryl (and hence R²) is a group of the formula:

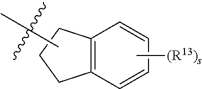

wherein R¹³ and s are as defined above.

In some embodiments r is 2 and the optionally substituted C₆-C₁₈aryl (and hence R²) is a group of the formula:

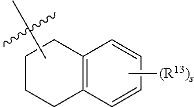

wherein R¹³ and s are as defined above.

In some embodiments s is selected from the group consisting of 0, 1, and 2. In some embodiments s is 0. In some embodiments s is 1. In some embodiments s is 2.

Each R¹³ substituent may be selected from any suitable optional substituent. In some embodiments each R¹³ is independently selected from the group consisting of H, F, CH₃, CH₂CH₃, OCH₃, CN, OCF₃, CO₂CH₃, NO₂, NH₂, NHCOCH₃, NHSO₂CH₃, NHCH₂CH₃, and CF₃.

In some embodiments R¹ is H, R³ is H, R⁶ is NH₂, R⁷ is H, X is (CH₂)ₘ wherein m is 0, and R² is a group of the formula:

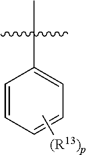

This provides compounds of formula (V):

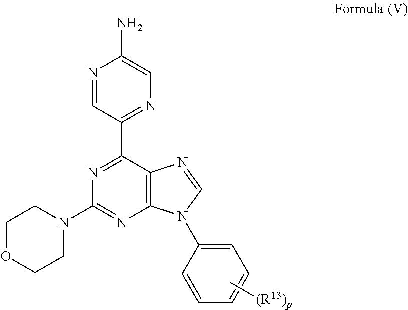

or a pharmaceutically acceptable salt or prodrug thereof,

wherein R¹³ and p are as defined above.

In some embodiments R¹ is H, R³ is H, R⁶ is NH₂, R⁷ is H, X is (CH₂)ₘ wherein m is 1, and R² is a group of the formula:

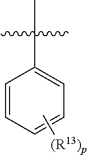

This provides compounds of formula (Va):

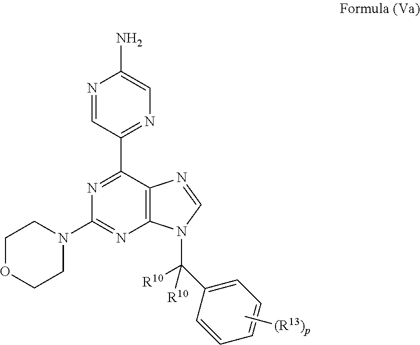

or a pharmaceutically acceptable salt or prodrug thereof,

wherein R¹⁰, R¹³ and p are as defined above.

In some embodiments of the compounds containing R¹⁰, R¹⁰ is selected from the group consisting of H, C₁-C₆haloalkyl, C₁-C₆hydroxyalkyl and C₁-C₆alkyl. In some embodiments R¹⁰ is selected from the group consisting of methyl, ethyl, propyl, isopropyl, and butyl. In some embodiments R¹⁰ is selected from the group consisting of H, methyl and ethyl.

In some embodiments R² is selected from the group consisting of cyano, optionally substituted C₁-C₁₂alkyl, and optionally substituted C₂-C₁₂heteroalkyl.

In some embodiments R² is selected from the group consisting of methyl, ethyl, isopropyl, propyl, 2-ethyl-propyl, 3,3-dimethyl-propyl, butyl, isobutyl, 3,3-dimethyl-butyl, 2-ethyl-butyl, pentyl, 2-methyl, pentyl, hexyl, heptyl, and octyl.

In some embodiments R² is an optionally substituted methyl group of the formula:

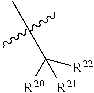

wherein R²⁰, R²¹ and R²² are each independently selected from the group consisting of H, Cl, Br, F, OH, NO₂, CN, NH₂, optionally substituted C₁-C₁₂alkyl and optionally substituted C₁-C₁₂heteroalkyl.

In some embodiments each R²⁰, R²¹ and R²² is independently selected from the group consisting of H, Cl, Br, F, OH, NO₂, CN, NH₂, methyl, ethyl, propyl, isopropyl, butyl, pentyl, methoxymethyl, 2-methoxyethyl, 3-methoxypropyl, 2-ethoxyethyl, 3-ethoxypropyl, aminomethyl, 2-aminoethyl, 3-aminopropyl, 4-aminobutyl, 5 aminopentyl, methylaminomethyl, 2-methylaminoethyl, 3-methylaminopropyl. 4-methylaminobutyl, 5-methylaminopentyl, ethylaminomethyl, 2-ethylaminoethyl, 3-ethylaminopropyl, 4-ethylaminobutyl, 5-ethylaminopentyl, dimethylaminomethyl, 2-dimethylaminoethyl, 3-dimethylaminopropyl, 4-dimethylaminobutyl, 5-dimethylaminopentyl, diethylaminomethyl, 2-diethylaminoethyl, 3-diethylaminopropyl, 4-diethylaminobutyl and 5-diethylaminopentyl.

In some embodiments R² is optionally substituted C₃-C₁₂cycloalkyl. In some embodiments R² is selected from the group consisting of optionally substituted cyclopropyl, optionally substituted cyclobutyl, optionally substituted cyclopentyl and optionally substituted cyclohexyl. In some embodiments R² is cyclopropyl.

In some embodiments R² is optionally substituted C₂-C₁₂heterocycloalkyl.

In some embodiments R² is selected from the group consisting of optionally substituted pyrrolidin-1-yl, optionally substituted pyrrolidin-2-yl, optionally substituted pyrrolidin-3-yl, optionally substituted dioxolane-2-yl, optionally substituted dioxolane-3-yl, optionally substituted tetrahydrofuran-2-yl, optionally substituted tetrahydrofuran-3-yl, optionally substituted piperidine-1-yl, optionally substituted piperidine-2-yl, optionally substituted piperidine-3-yl, optionally substituted piperidine-4-yl, optionally substituted morpholine-1-yl, optionally substituted morpholine-2-yl, optionally substituted morpholine-3-yl, optionally substituted 1,4,dioxolane-2-yl, optionally substituted thiomorpholine-1-yl, optionally substituted thiomorpholine-2-yl, optionally substituted thiomorpholine-3-yl, optionally substituted thiomorpholine-4-yl, optionally substituted piperazine-1-yl and optionally substituted piperazine-2-yl.

In some embodiments the optionally substituted C₂-C₁₂heterocycloalkyl group is selected from the group consisting of:

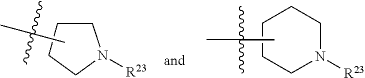

wherein R²³ is independently selected from the group consisting of H, optionally substituted C₁-C₁₂alkyl, optionally substituted C₂-C₁₂alkenyl, optionally substituted C₂-C₁₂alkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈heteroaryl, optionally substituted C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁₈heteroaryloxy, optionally substituted C₁-C₁₂alkylamino, SO₂NR²⁴R²⁵, SOR²⁴, SO₂R²⁴, SONR²⁴R²⁵, SOR²⁴, COR²⁴, COOH, COOR²⁴, and CONR²⁴R²⁵;

each R²⁴ and R²⁵ is independently selected from the group consisting of H, optionally substituted C₁-C₁₂alkyl, optionally substituted C₂-C₁₂alkenyl, optionally substituted C₂-C₁₂alkynyl, optionally substituted C₂-C₁₀heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, and optionally substituted C₁-C₁₈heteroaryl.

In some embodiments the optionally substituted C₂-C₁₂heterocycloalkyl group is selected from the group consisting of:

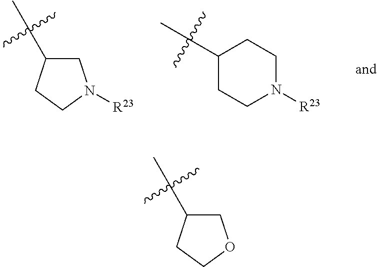

wherein R²³ is as defined above.

In some embodiments R²³ is selected from the group consisting of H, COR²⁴, and COOR²⁴.

In some embodiments R²⁴ is selected from the group consisting of H, optionally substituted C₁-C₁₂alkyl, optionally substituted C₆-C₁₈aryl, and optionally substituted C₁-C₁₈heteroaryl. In some embodiments R²⁴ is C₁-C₆ alkyl. In some embodiments R²⁴ is methyl.

In some embodiments R² is an optionally substituted C₂-C₁₂heteroalkyl group. In some embodiments the C₂-C₁₂heteroalkyl group is selected from the group consisting of hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl. Examples of possible values of R² as C₂-C₁₂heteroalkyl include hydroxymethyl, hydroxyethyl, hydroxypropyl, hydroxybutyl, hydroxypentyl, methoxymethyl, 2-methoxyethyl, 3-methoxypropyl, 2-ethoxyethyl, 3-ethoxypropyl, aminomethyl, 2-aminoethyl, 3-aminopropyl, 4-aminobutyl, 5 aminopentyl, methylaminomethyl, 2-methylaminoethyl, 3-methylaminopropyl, 4-methylaminobutyl, 5-methylaminopentyl, ethylaminomethyl, 2-ethylaminoethyl, 3-ethylaminopropyl, 4-ethylaminobutyl, 5-ethylaminopentyl, dimethylaminomethyl, 2-dimethylaminoethyl, 3-dimethylaminopropyl, 4-dimethylaminobutyl, 5-dimethylaminopentyl, diethylaminomethyl, 2-diethylaminoethyl, 3-diethylaminopropyl, 4-diethylaminobutyl and 5-diethylaminopentyl.

In some embodiments R² is selected from the group consisting of methyl, ethyl, isopropyl, propyl, 2-ethyl-propyl, 3,3-dimethyl-propyl, cyclopropyl, cyclopentyl, butyl, isobutyl, 3,3-dimethyl-butyl, 2-ethyl-butyl, pentyl, 2-methyl, pentyl, hexyl, heptyl, octyl, cyano, methoxymethyl and butoxymethyl.

Many if not all of the variables discussed above may be optionally substituted. If the variable is optionally substituted then in some embodiments each optional substituent is independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)R^{a}, —C(═O)OR^{a}, C(═O)NR^{a}R^{b}, C(═NOH)R^{a}, C(═NR^{a})NR^{b}R^{c}, NR^{a}R^{b}, NR^{a}C(═O)R^{b}, NR^{a}C(═O)OR^{b}, NR^{a}C(═O)NR^{b}R^{c}, NR^{a}C(═NR^{b})NR^{c}R^{d}, NR^{a}SO₂R^{b}, —SR^{a}, SO₂NR^{a}R^{b}, —OR^{a}, OC(═O)NR^{a}R^{b}, OC(═O)R^{a} and acyl,

wherein R^{a}, R^{b}, R^{c} and R^{d} are each independently selected from the group consisting of H, C₁-C₁₂alkyl, C₁-C₁₂haloalkyl, C₂-C₁₂alkenyl, C₂-C₁₂alkynyl, C₁-C₁₀ heteroalkyl, C₃-C₁₂cycloalkyl, C₃-C₁₂cycloalkenyl, C₁-C₁₂heterocycloalkyl, C₁-C₁₂ heterocycloalkenyl, C₆-C₁₈aryl, C₁-C₁₈heteroaryl, and acyl, or any two or more of R^{a}, R^{b}, R^{c} and R^{d}, when taken together with the atoms to which they are attached form a heterocyclic ring system with 3 to 12 ring atoms.

In some embodiments each optional substituent is independently selected from the group consisting of: F, Cl, Br, ═O, ═S, —CN, —NO₂, alkyl, alkenyl, heteroalkyl, haloalkyl, alkynyl, aryl, cycloalkyl, heterocycloalkyl, heteroaryl, hydroxy, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR^{a}, COOH, SH, and acyl.

In some embodiments each optional substituent is independently selected from the group consisting of: F, Br, Cl, ═O, ═S, —CN methyl, trifluoro-methyl, ethyl, 2,2,2-trifluoroethyl, isopropyl, propyl, 2-ethyl-propyl, 3,3-dimethyl-propyl, butyl, isobutyl, 3,3-dimethyl-butyl, 2-ethyl-butyl, pentyl, 2-methyl-pentyl, pent-4-enyl, hexyl, heptyl, octyl, phenyl, NH₂, —NO₂, phenoxy, hydroxy, methoxy, trifluoro-methoxy, ethoxy, and methylenedioxy.

In addition to compounds of formula I, the embodiments disclosed are also directed to pharmaceutically acceptable salts, pharmaceutically acceptable N-oxides, pharmaceutically acceptable prodrugs, and pharmaceutically active metabolites of such compounds, and pharmaceutically acceptable salts of such metabolites.

The invention also relates to pharmaceutical compositions including a compound of the invention and a pharmaceutically acceptable carrier, diluent or excipient.

In a further aspect the invention provides a method of inhibiting a protein kinase selected from the group consisting of a serine/threonine protein kinase or a fragment or a complex thereof or a functional equivalent thereof and a PI3 kinase or a fragment or a complex thereof or a functional equivalent thereof, the method including exposing the protein kinase or a fragment or complex thereof or a functional equivalent thereof and/or co-factor(s) thereof to an effective amount of a compound according to formula (I) as described herein.

The compounds disclosed herein may act directly and solely on the kinase molecule or a complex or fragment thereof to inhibit biological activity. However, it is understood that the compounds may also act at least partially on co-factors that are involved in the phosphorylation process. Known kinase co-factors include ionic species (such as zinc and calcium), lipids (such as phosphatidylserine), and diacylglycerols.

In some embodiments the protein kinase is a serine/threonine protein kinase or a fragment or a complex thereof or a functional equivalent thereof. In some embodiments the serine/threonine protein kinase or a fragment or complex thereof is an mTOR protein kinase or a fragment thereof, or a complex thereof or a functional equivalent thereof. In some embodiments the serine/threonine protein kinase is mTORC1 or a fragment or complex thereof or a functional equivalent thereof.

In some embodiments the protein kinase is a PI3 kinase or a fragment thereof or a complex thereof or a functional equivalent thereof. In some embodiments the PI3 kinase or a fragment thereof or a complex thereof or a functional equivalent thereof, is a class I PI3K or a fragment thereof or a complex thereof or a functional equivalent thereof.

In one embodiment of the method exposing the one or more protein kinase(s) to the compound includes administering the compound to a mammal containing the one or more protein kinase(s).

In an even further aspect the invention provides the use of a compound of formula (I) to inhibit one or more protein kinase(s) selected from the group consisting of a serine/threonine protein kinase or a fragment or a complex thereof or a functional equivalent thereof and a PI3 kinase or a fragment or a complex thereof or a functional equivalent thereof.

In some embodiments the protein kinase is a serine/threonine protein kinase or a fragment or a complex thereof or a functional equivalent thereof. In some embodiments the serine/threonine protein kinase or a fragment or complex thereof is an mTOR protein kinase or a fragment thereof, or a complex thereof or a functional equivalent thereof. In some embodiments the serine/threonine protein kinase is mTORC1 or a fragment or complex thereof or a functional equivalent thereof.

In some embodiments the protein kinase is a PI3 kinase or a fragment thereof or a complex thereof or a functional equivalent thereof. In some embodiments the PI3 kinase or a fragment thereof or a complex thereof or a functional equivalent thereof, is a class I PI3K or a fragment thereof or a complex thereof or a functional equivalent thereof.

In an even further aspect the invention provides a method of treating or preventing a condition in a mammal in which inhibition of one or more protein kinase(s) selected from the group consisting of a serine/threonine protein kinase or a fragment or a complex thereof or a functional equivalent thereof and a PI3 kinase or a fragment or a complex thereof or a functional equivalent thereof, prevents, inhibits or ameliorates a pathology or a symptomology of the condition, the method including administration of a therapeutically effective amount of a compound of formula (I).

In some embodiments the protein kinase is a serine/threonine protein kinase or a fragment or a complex thereof or a functional equivalent thereof. In some embodiments the serine/threonine protein kinase or a fragment or complex thereof is an mTOR protein kinase or a fragment thereof, or a complex thereof or a functional equivalent thereof. In some embodiments the serine/threonine protein kinase is mTORC1 or a fragment or complex thereof or a functional equivalent thereof.

In some embodiments the protein kinase is a PI3 kinase or a fragment thereof or a complex thereof or a functional equivalent thereof. In some embodiments the PI3 kinase or a fragment thereof or a complex thereof or a functional equivalent thereof, is a class I PI3K or a fragment thereof or a complex thereof or a functional equivalent thereof.

In some embodiments the condition is cancer. In some embodiments the cancer is selected from the group consisting of Hematologic cancer such as myeloproliferative disorders (idiopathic myelofibrosis, polycythemia vera, essential thrombocythemia, chronic myeloid leukemia), myeloid metaplasia, chronic myelomonocytic leukemia, acute lymphocytic leukemia, acute erythroblastic leukemia, Hodgkin's and Non Hodgkin's disease, B-cell lymphoma, acute T-cell leukemia, myelodysplastic syndromes, plasma cell disorder, hairy cell leukemia, kaposi's sarcoma, lymphoma; gynaecologic cancer such as breast carcinoma, ovarian cancer, cervical cancer, vaginal and vulva cancer, endometrial hyperplasia; gastrointestinal tract cancer such as colorectal carcinoma, polyps, liver cancer, gastric cancer, pancreatic cancer, gall bladder cancer; urinary tract cancer such as prostate cancer, kidney and renal cancer; urinary bladder cancer, urethral cancer, penile cancer; skin cancer such as melanoma; brain tumour such as glioblastoma, neuroblastoma, astrocytoma, ependynoma, brain-stem gliomas, medulloblastoma, menigiomas, astrocytoma, oligodendroglioma; head and neck cancer such as nasopharyngeal carcinoma, laryngeal carcinoma; respiratory tract cancer such as lung carcinoma (NSCLC and SCLC), mesothelioma; eye disease such as retinoblastoma; musculo-skeleton diseases such as osteosarcoma, musculoskeleletal neoplasm; Squamous cell carcinoma and fibroid tumour.

In an even further aspect the invention provides use of a compound of formula (I) in the preparation of a medicament for treating a condition in an animal in which inhibition of one or more protein kinase(s) selected from the group consisting of a serine/threonine protein kinase or a fragment or a complex thereof or a functional equivalent thereof and a PI3 kinase or a fragment or a complex thereof or a functional equivalent thereof, prevents, inhibits or ameliorates a pathology or a symptomology of the condition.

In another aspect the present invention provides the use of a compound of formula (I) or a pharmaceutically acceptable salt, N-oxide or prodrug thereof in the treatment of a condition in which inhibition of one or more protein kinase(s) selected from the group consisting of a serine/threonine protein kinase or a fragment or a complex thereof or a functional equivalent thereof and a PI3 kinase or a fragment or a complex thereof or a functional equivalent thereof, prevents, inhibits or ameliorates a pathology or a symptomology of the condition.

In another aspect the present invention provides a method of prevention or treatment of a proliferative condition in a subject, the method including administration of a therapeutically effective amount of a compound of formula (I).

In another aspect the present invention provides the use of a compound of formula (I) in the preparation of a medicament for treating a proliferative condition in a subject.

In some embodiments the protein kinase is a serine/threonine protein kinase or a fragment or a complex thereof or a functional equivalent thereof. In some embodiments the serine/threonine protein kinase or a fragment or complex thereof is an mTOR protein kinase or a fragment thereof, or a complex thereof or a functional equivalent thereof. In some embodiments the serine/threonine protein kinase is mTORC1 or a fragment or complex thereof or a functional equivalent thereof.

In some embodiments the protein kinase is a PI3 kinase or a fragment thereof or a complex thereof or a functional equivalent thereof. In some embodiments the PI3 kinase or a fragment thereof or a complex thereof or a functional equivalent thereof, is a class I PI3K or a fragment thereof or a complex thereof or a functional equivalent thereof.

In some embodiments the condition is cancer. In some embodiments the cancer is selected from the group consisting of Hematologic cancer such as myeloproliferative disorders (idiopathic myelofibrosis, polycythemia vera, essential thrombocythemia, chronic myeloid leukemia), myeloid metaplasia, chronic myelomonocytic leukemia, acute lymphocytic leukemia, acute erythroblastic leukemia, Hodgkin's and Non Hodgkin's disease, B-cell lymphoma, acute T-cell leukemia, myelodysplastic syndromes, plasma cell disorder, hairy cell leukemia, kaposi's sarcoma, lymphoma; gynaecologic cancer such as breast carcinoma, ovarian cancer, cervical cancer, vaginal and vulva cancer, endometrial hyperplasia; gastrointestinal tract cancer such as colorectal carcinoma, polyps, liver cancer, gastric cancer, pancreatic cancer, gall bladder cancer; urinary tract cancer such as prostate cancer, kidney and renal cancer; urinary bladder cancer, urethral cancer, penile cancer; skin cancer such as melanoma; brain tumour such as glioblastoma, neuroblastoma, astrocytoma, ependynoma, brain-stem gliomas, medulloblastoma, menigiomas, astrocytoma, oligodendroglioma; head and neck cancer such as nasopharyngeal carcinoma, laryngeal carcinoma; respiratory tract cancer such as lung carcinoma (NSCLC and SCLC), mesothelioma; eye disease such as retinoblastoma; musculo-skeleton diseases such as osteosarcoma, musculoskeleletal neoplasm; Squamous cell carcinoma and fibroid tumour.

These and other features of the present teachings are set forth herein.

### DETAILED DESCRIPTION

In this specification a number of terms are used which are well known to a skilled addressee. Nevertheless for the purposes of clarity a number of terms will be defined.

As used herein, the term “unsubstituted” means that there is no substituent or that the only substituents are hydrogen.

The term “optionally substituted” as used throughout the specification denotes that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)R^{a}, —C(═O)OR^{a}, C(═O)NR^{a}R^{b}, C(═NOH)R^{a}, C(═NR^{a})NR^{b}R^{c}, NR^{a}R^{b}, NR^{a}C(═O)R^{b}, NR^{a}C(═O)OR^{b}, NR^{a}C(═O)NR^{b}R^{c}, NR^{a}C(═NR^{b})NR^{c}R^{d}, NR^{a}SO₂R^{b}, —SR^{a}, SO₂NR^{a}R^{b}, —OR^{a}, OC(═O)NR^{a}R^{b}, OC(═O)R^{a} and acyl,

wherein R^{a}, R^{b}, R^{c} and R^{d} are each independently selected from the group consisting of H, C₁-C₁₂alkyl, C₁-C₁₂haloalkyl, C₂-C₁₂alkenyl, C₂-C₁₂alkynyl, C₂-C₁₀ heteroalkyl, C₃-C₁₂cycloalkyl, C₃-C₁₂cycloalkenyl, C₂-C₁₂heterocycloalkyl, C₂-C₁₂ heterocycloalkenyl, C₆-C₁₈aryl, C₁-C₁₈heteroaryl, and acyl, or any two or more of R^{a}, R^{b}, R^{c} and R^{d}, when taken together with the atoms to which they are attached form a heterocyclic ring system with 3 to 12 ring atoms.

In some embodiments each optional substituent is independently selected from the group consisting of: halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, —COOH, —SH, and acyl.

Examples of particularly suitable optional substituents include F, Cl, Br, I, CH₃, CH₂CH₃, OH, OCH₃, CF₃, OCF₃, NO₂, NH₂, and CN.

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

“Acyl” means an R—C(═O)— group in which the R group may be an alkyl, cycloalkyl, heterocycloalkyl, aryl or heteroaryl group as defined herein. Examples of acyl include acetyl and benzoyl. The group may be a terminal group or a bridging group. If the group is a terminal group it is bonded to the remainder of the molecule through the carbonyl carbon.

“Acylamino” means an R—C(═O)—NH— group in which the R group may be an alkyl, cycloalkyl, heterocycloalkyl, aryl or heteroaryl group as defined herein. The group may be a terminal group or a bridging group. If the group is a terminal group it is bonded to the remainder of the molecule through the nitrogen atom.

“Alkenyl” as a group or part of a group denotes an aliphatic hydrocarbon group containing at least one carbon-carbon double bond and which may be straight or branched preferably having 2-12 carbon atoms, more preferably 2-10 carbon atoms, most preferably 2-6 carbon atoms, in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl. The group may be a terminal group or a bridging group.

“Alkenyloxy” refers to an alkenyl-O— group in which alkenyl is as defined herein. Preferred alkenyloxy groups are C₁-C₆ alkenyloxy groups. The group may be a terminal group or a bridging group. If the group is a terminal group it is bonded to the remainder of the molecule through the oxygen atom.

“Alkyl” as a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both mono-alkylamino and dialkylamino, unless specified. “Mono-alkylamino” means a Alkyl-NH— group, in which alkyl is as defined herein. “Dialkylamino” means a (alkyl)₂N— group, in which each alkyl may be the same or different and are each as defined herein for alkyl. The alkyl group is preferably a C₁-C₆ alkyl group. The group may be a terminal group or a bridging group. If the group is a terminal group it is bonded to the remainder of the molecule through the nitrogen atom.

“Alkylaminocarbonyl” refers to a group of the formula (Alkyl)ₓ(H)_{y}NC(═O)— in which alkyl is as defined herein, x is 1 or 2, and the sum of X+Y=2. The group may be a terminal group or a bridging group. If the group is a terminal group it is bonded to the remainder of the molecule through the carbonyl carbon.

“Alkyloxy” refers to an alkyl-O— group in which alkyl is as defined herein. Preferably the alkyloxy is a C₁-C₆alkyloxy. Examples include, but are not limited to, methoxy and ethoxy. The group may be a terminal group or a bridging group.

“Alkyloxyalkyl” refers to an alkyloxy-alkyl-group in which the alkyloxy and alkyl moieties are as defined herein. The group may be a terminal group or a bridging group. If the group is a terminal group it is bonded to the remainder of the molecule through the alkyl group.

“Alkyloxyaryl” refers to an alkyloxy-aryl-group in which the alkyloxy and aryl moieties are as defined herein. The group may be a terminal group or a bridging group. If the group is a terminal group it is bonded to the remainder of the molecule through the aryl group.

“Alkyloxycarbonyl” refers to an alkyl-O—C(═O)— group in which alkyl is as defined herein. The alkyl group is preferably a C₁-C₆ alkyl group. Examples include, but are not limited to, methoxycarbonyl and ethoxycarbonyl. The group may be a terminal group or a bridging group. If the group is a terminal group it is bonded to the remainder of the molecule through the carbonyl carbon.

“Alkyloxycycloalkyl” refers to an alkyloxy-cycloalkyl-group in which the alkyloxy and cycloalkyl moieties are as defined herein. The group may be a terminal group or a bridging group. If the group is a terminal group it is bonded to the remainder of the molecule through the cycloalkyl group.

“Alkyloxyheteroaryl” refers to an alkyloxy-heteroaryl-group in which the alkyloxy and heteroaryl moieties are as defined herein. The group may be a terminal group or a bridging group. If the group is a terminal group it is bonded to the remainder of the molecule through the heteroaryl group.

“Alkyloxyheterocycloalkyl” refers to an alkyloxy-heterocycloalkyl-group in which the alkyloxy and heterocycloalkyl moieties are as defined herein. The group may be a terminal group or a bridging group. If the group is a terminal group it is bonded to the remainder of the molecule through the heterocycloalkyl group.

“Alkylsulfinyl” means an alkyl-S—(═O)— group in which alkyl is as defined herein. The alkyl group is preferably a C₁-C₆ alkyl group. Exemplary alkylsulfinyl groups include, but not limited to, methylsulfinyl and ethylsulfinyl. The group may be a terminal group or a bridging group. If the group is a terminal group it is bonded to the remainder of the molecule through the sulfur atom.

“Alkylsulfonyl” refers to an alkyl-S(═O)₂— group in which alkyl is as defined above. The alkyl group is preferably a C₁-C₆alkyl group. Examples include, but not limited to methylsulfonyl and ethylsulfonyl. The group may be a terminal group or a bridging group. If the group is a terminal group it is bonded to the remainder of the molecule through the sulfur atom.

“Alkynyl” as a group or part of a group means an aliphatic hydrocarbon group containing a carbon-carbon triple bond and which may be straight or branched preferably having from 2-12 carbon atoms, more preferably 2-10 carbon atoms, more preferably 2-6 carbon atoms in the normal chain. Exemplary structures include, but are not limited to, ethynyl and propynyl. The group may be a terminal group or a bridging group.

“Alkynyloxy” refers to an alkynyl-O— group in which alkynyl is as defined herein. Preferred alkynyloxy groups are C₁-C₆alkynyloxy groups. The group may be a terminal group or a bridging group. If the group is a terminal group it is bonded to the remainder of the molecule through the oxygen atom.

“Aminoalkyl” means an NH₂-alkyl-group in which the alkyl group is as defined herein. The group may be a terminal group or a bridging group. If the group is a terminal group it is bonded to the remainder of the molecule through the alkyl group.

“Aminosulfonyl” means an NH₂—S(═O)₂— group. The group may be a terminal group or a bridging group. If the group is a terminal group it is bonded to the remainder of the molecule through the sulfur atom.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group. Typically an aryl group is a C₆-C₁₈ aryl group.

“Arylalkenyl” means an aryl-alkenyl-group in which the aryl and alkenyl are as defined herein. Exemplary arylalkenyl groups include phenylallyl. The group may be a terminal group or a bridging group. If the group is a terminal group it is bonded to the remainder of the molecule through the alkenyl group.

“Arylalkyl” means an aryl-alkyl-group in which the aryl and alkyl moieties are as defined herein. Preferred arylalkyl groups contain a C₁₋₅alkyl moiety. Exemplary arylalkyl groups include benzyl, phenethyl, 1-naphthalenemethyl and 2-naphthalenemethyl. The group may be a terminal group or a bridging group. If the group is a terminal group it is bonded to the remainder of the molecule through the alkyl group.

“Arylalkyloxy” refers to an aryl-alkyl-O— group in which the alkyl and aryl are as defined herein. The group may be a terminal group or a bridging group. If the group is a terminal group it is bonded to the remainder of the molecule through the oxygen atom.

“Arylamino” includes both mono-arylamino and di-arylamino unless specified. Mono-arylamino means a group of formula arylNH—, in which aryl is as defined herein. di-arylamino means a group of formula (aryl)₂N— where each aryl may be the same or different and are each as defined herein for aryl. The group may be a terminal group or a bridging group. If the group is a terminal group it is bonded to the remainder of the molecule through the nitrogen atom.

“Arylheteroalkyl” means an aryl-heteroalkyl-group in which the aryl and heteroalkyl moieties are as defined herein. The group may be a terminal group or a bridging group. If the group is a terminal group it is bonded to the remainder of the molecule through the heteroalkyl group.

“Aryloxy” refers to an aryl-O— group in which the aryl is as defined herein. Preferably the aryloxy is a C₆-C₁₈aryloxy, more preferably a C₆-C₁₀aryloxy. The group may be a terminal group or a bridging group. If the group is a terminal group it is bonded to the remainder of the molecule through the oxygen atom.

“Arylsulfonyl” means an aryl-S(═O)₂— group in which the aryl group is as defined herein. The group may be a terminal group or a bridging group. If the group is a terminal group it is bonded to the remainder of the molecule through the sulfur atom.

A “bond” is a linkage between atoms in a compound or molecule. The bond may be a single bond, a double bond, or a triple bond.

“Cycloalkenyl” means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups. A cycloalkenyl group typically is a C₃-C₁₂ alkenyl group. The group may be a terminal group or a bridging group.

“Cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₁₂ alkyl group. The group may be a terminal group or a bridging group.

“Cycloalkylalkyl” means a cycloalkyl-alkyl-group in which the cycloalkyl and alkyl moieties are as defined herein. Exemplary monocycloalkylalkyl groups include cyclopropylmethyl, cyclopentylmethyl, cyclohexylmethyl and cycloheptylmethyl. The group may be a terminal group or a bridging group. If the group is a terminal group it is bonded to the remainder of the molecule through the alkyl group.

“Cycloalkylalkenyl” means a cycloalkyl-alkenyl-group in which the cycloalkyl and alkenyl moieties are as defined herein. The group may be a terminal group or a bridging group. If the group is a terminal group it is bonded to the remainder of the molecule through the alkenyl group.

“Cycloalkylheteroalkyl” means a cycloalkyl-heteroalkyl-group in which the cycloalkyl and heteroalkyl moieties are as defined herein. The group may be a terminal group or a bridging group. If the group is a terminal group it is bonded to the remainder of the molecule through the heteroalkyl group.

“Cycloalkyloxy” refers to a cycloalkyl-O— group in which cycloalkyl is as defined herein. Preferably the cycloalkyloxy is a C₁-C₆cycloalkyloxy. Examples include, but are not limited to, cyclopropanoxy and cyclobutanoxy. The group may be a terminal group or a bridging group. If the group is a terminal group it is bonded to the remainder of the molecule through the oxygen atom.

“Cycloalkenyloxy” refers to a cycloalkenyl-O— group in which the cycloalkenyl is as defined herein. Preferably the cycloalkenyloxy is a C₁-C₆cycloalkenyloxy. The group may be a terminal group or a bridging group. If the group is a terminal group it is bonded to the remainder of the molecule through the oxygen atom.

“Haloalkyl” refers to an alkyl group as defined herein in which one or more of the hydrogen atoms has been replaced with a halogen atom selected from the group consisting of fluorine, chlorine, bromine and iodine. A haloalkyl group typically has the formula CₙH₍₂ₙ₊₁₋ₘ₎Xₘ wherein each X is independently selected from the group consisting of F, Cl, Br and I. In groups of this type n is typically from 1 to 10, more preferably from 1 to 6, most preferably 1 to 3. m is typically 1 to 6, more preferably 1 to 3. Examples of haloalkyl include fluoromethyl, difluoromethyl and trifluoromethyl.

“Haloalkenyl” refers to an alkenyl group as defined herein in which one or more of the hydrogen atoms has been replaced with a halogen atom independently selected from the group consisting of F, Cl, Br and I.

“Haloalkynyl” refers to an alkynyl group as defined herein in which one or more of the hydrogen atoms has been replaced with a halogen atom independently selected from the group consisting of F, Cl, Br and I.

“Halogen” represents chlorine, fluorine, bromine or iodine.

“Heteroalkyl” refers to a straight- or branched-chain alkyl group preferably having from 2 to 12 carbons, more preferably 2 to 6 carbons in the chain, in which one or more of the carbon atoms (and any associated hydrogen atoms) are each independently replaced by a heteroatomic group selected from S, O, P and NR′ where R′ is selected from the group consisting of H, optionally substituted C₁-C₁₂alkyl, optionally substituted C₃-C₁₂cycloalkyl, optionally substituted C₆-C₁₈aryl, and optionally substituted C₁-C₁₈heteroaryl. Exemplary heteroalkyls include alkyl ethers, secondary and tertiary alkyl amines, amides, alkyl sulfides, and the like. Examples of heteroalkyl also include hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl. The group may be a terminal group or a bridging group.

“Heteroalkyloxy” refers to an heteroalkyl-O— group in which heteroalkyl is as defined herein. Preferably the heteroalkyloxy is a C₂-C₆heteroalkyloxy. The group may be a terminal group or a bridging group.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, 1,3,5-triazene, tetrazole, indole, isoindole, 1H-indazole, benzotriazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, 1,2,3-oxadiazole, 1,2,4-oxadiazole, 1,2,3-triazole, 1,2,4-triazole, 1,2,4-thiadiazole, 1,3,4-thiadiazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. A heteroaryl group is typically a C₁-C₁₈ heteroaryl group. The group may be a terminal group or a bridging group.

“Heteroarylalkyl” means a heteroaryl-alkyl group in which the heteroaryl and alkyl moieties are as defined herein. Preferred heteroarylalkyl groups contain a lower alkyl moiety. Exemplary heteroarylalkyl groups include pyridylmethyl. The group may be a terminal group or a bridging group. If the group is a terminal group it is bonded to the remainder of the molecule through the alkyl group.

“Heteroarylalkenyl” means a heteroaryl-alkenyl-group in which the heteroaryl and alkenyl moieties are as defined herein. The group may be a terminal group or a bridging group. If the group is a terminal group it is bonded to the remainder of the molecule through the alkenyl group.

“Heteroarylheteroalkyl” means a heteroaryl-heteroalkyl-group in which the heteroaryl and heteroalkyl moieties are as defined herein. The group may be a terminal group or a bridging group. If the group is a terminal group it is bonded to the remainder of the molecule through the heteroalkyl group.

“Heteroaryloxy” refers to a heteroaryl-O— group in which the heteroaryl is as defined herein. Preferably the heteroaryloxy is a C₁-C₁₈heteroaryloxy. The group may be a terminal group or a bridging group. If the group is a terminal group it is bonded to the remainder of the molecule through the oxygen atom.

“Heterocyclic” refers to saturated, partially unsaturated or fully unsaturated monocyclic, bicyclic or polycyclic ring system containing at least one heteroatom selected from the group consisting of nitrogen, sulfur and oxygen as a ring atom. Examples of heterocyclic moieties include heterocycloalkyl, heterocycloalkenyl and heteroaryl.

“Heterocycloalkenyl” refers to a heterocycloalkyl group as defined herein but containing at least one double bond. A heterocycloalkenyl group typically is a C₂-C₁₂ heterocycloalkenyl group. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. A heterocycloalkyl group typically is a C₂-C₁₂ heterocycloalkyl group. The group may be a terminal group or a bridging group.

“Heterocycloalkylalkyl” refers to a heterocycloalkyl-alkyl-group in which the heterocycloalkyl and alkyl moieties are as defined herein. Exemplary heterocycloalkylalkyl groups include (2-tetrahydrofuryl)methyl, (2-tetrahydrothiofuranyl)methyl. The group may be a terminal group or a bridging group. If the group is a terminal group it is bonded to the remainder of the molecule through the alkyl group.

“Heterocycloalkylalkenyl” refers to a heterocycloalkyl-alkenyl-group in which the heterocycloalkyl and alkenyl moieties are as defined herein. The group may be a terminal group or a bridging group. If the group is a terminal group it is bonded to the remainder of the molecule through the alkenyl group.

“Heterocycloalkylheteroalkyl” means a heterocycloalkyl-heteroalkyl-group in which the heterocycloalkyl and heteroalkyl moieties are as defined herein. The group may be a terminal group or a bridging group. If the group is a terminal group it is bonded to the remainder of the molecule through the heteroalkyl group.

“Heterocycloalkyloxy” refers to a heterocycloalkyl-O— group in which the heterocycloalkyl is as defined herein. Preferably the heterocycloalkyloxy is a C₁-C₆heterocycloalkyloxy. The group may be a terminal group or a bridging group. If the group is a terminal group it is bonded to the remainder of the molecule through the oxygen atom.

“Heterocycloalkenyloxy” refers to a heterocycloalkenyl-O— group in which heterocycloalkenyl is as defined herein. Preferably the Heterocycloalkenyloxy is a C₁-C₆ Heterocycloalkenyloxy. The group may be a terminal group or a bridging group. If the group is a terminal group it is bonded to the remainder of the molecule through the oxygen atom.

“Hydroxyalkyl” refers to an alkyl group as defined herein in which one or more of the hydrogen atoms has been replaced with an OH group. A hydroxyalkyl group typically has the formula CₙH₍₂ₙ₊₁₋ₓ₎(OH)ₓ. In groups of this type n is typically from 1 to 10, more preferably from 1 to 6, most preferably 1 to 3. x is typically 1 to 6, more preferably 1 to 3.

“Lower alkyl” as a group means unless otherwise specified, an aliphatic hydrocarbon group which may be straight or branched having 1 to 6 carbon atoms in the chain, more preferably 1 to 4 carbons such as methyl, ethyl, propyl (n-propyl or isopropyl) or butyl (n-butyl, isobutyl or tertiary-butyl). The group may be a terminal group or a bridging group.

“Sulfinyl” means an R—S(═O)— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfinylamino” means an R—S(═O)—NH— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the nitrogen atom.

“Sulfonyl” means an R—S(═O)₂— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfonylamino” means an R—S(═O)₂—NH— group. The group may be a terminal group or a bridging group. If the group is a terminal group it is bonded to the remainder of the molecule through the nitrogen atom.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

“Prodrug” means a compound that undergoes conversion to a compound of formula (I) within a biological system, usually by metabolic means (e.g. by hydrolysis, reduction or oxidation). For example an ester prodrug of a compound of formula (I) containing a hydroxyl group may be convertible by hydrolysis in vivo to the parent molecule. Suitable esters of compounds of formula (I) containing a hydroxyl group, are for example acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsulphamates and quinates. As another example an ester prodrug of a compound of formula (I) containing a carboxy group may be convertible by hydrolysis in vivo to the parent molecule. (Examples of ester prodrugs are those described by F. J. Leinweber, Drug Metab. Res., 18:379, 1987). Similarly, an acyl prodrug of a compound of formula (I) containing an amino group may be convertible by hydrolysis in vivo to the parent molecule. (Many examples of prodrugs for these and other functional groups, including amines, are described in Prodrugs: Challenges and Rewards (Parts 1 and 2); Ed V. Stella, R. Borchardt, M. Hageman, R. Oliyai, H. Maag and J Tilley; Springer, 2007.)

The term “oxygen protecting group” means a group that can prevent the oxygen moiety reacting during further derivatisation of the protected compound and which can be readily removed when desired. In one embodiment the protecting group is removable in the physiological state by natural metabolic processes and in essence the protected compound is acting as a prodrug for the active unprotected species. Examples of oxygen protecting groups include acyl groups (such as acetyl), ethers (such as methoxy methyl ether (MOM), B-methoxy ethoxy methyl ether (MEM), p-methoxy benzyl ether (PMB), methylthio methyl ether, Pivaloyl (Piv), Tetrahydropyran (THP)), and silyl ethers (such as Trimethylsilyl (TMS) tert-butyl dimethyl silyl (TBDMS) and triisopropylsilyl (TIPS).

The term “nitrogen protecting group” means a group that can prevent the nitrogen moiety reacting during further derivatisation of the protected compound and which can be readily removed when desired. In one embodiment the protecting group is removable in the physiological state by natural metabolic processes and in essence the protected compound is acting as a prodrug for the active unprotected species. Examples of suitable nitrogen protecting groups that may be used include formyl, trityl, phthalimido, acetyl, trichloroacetyl, chloroacetyl, bromoacetyl, iodoacetyl; urethane-type blocking groups such as benzyloxycarbonyl (‘CBz’), 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, t-butoxycarbonyl (‘tBoc’), 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluoyl)-prop-2-yloxy-carbonyl, cyclo-pentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfono)-ethoxycarbonyl, 2-(methylsulfono)ethoxycarbonyl, 2-(triphenylphosphino)-ethoxycarbonyl, fluorenylmethoxycarbonyl (“FMOC”), 2-(trimethylsilyl)ethoxycarbonyl, allyloxycarbonyl, 1-(trimethylsilylmethyl)prop-1-enyloxycarbonyl, 5-benzisoxalymethoxy carbonyl, 4-acetoxybenzyloxycarbonyl, 2,2,2-trichloroethoxycarbonyl, 2-ethynyl-2-propoxycarbonyl, cyclopropylmethoxycarbonyl, 4-(decycloxy)benzyloxycarbonyl, isobornyloxycarbonyl, 1-piperidyloxycarbonlyl and the like; benzoylmethylsulfono group, 2-nitrophenylsulfenyl, diphenylphosphine oxide, and the like. The actual nitrogen protecting group employed is not critical so long as the derivatised nitrogen group is stable to the condition of subsequent reaction(s) and can be selectively removed as required without substantially disrupting the remainder of the molecule including any other nitrogen protecting group(s). Further examples of these groups are found in: Greene, T. W. and Wuts, P. G. M., Protective Groups in Organic Synthesis, Second edition; Wiley-Interscience: 1991; Chapter 7; McOmie, J. F. W. (ed.), Protective Groups in Organic Chemistry, Plenum Press, 1973; and Kocienski, P. J., Protecting Groups, Second Edition, Theime Medical Pub., 2000.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

The term “functional equivalent” is intended to include variants of the specific protein kinase species described herein. It will be understood that kinases may have isoforms, such that while the primary, secondary, tertiary or quaternary structure of a given kinase isoform is different to the protoypical kinase, the molecule maintains biological activity as a protein kinase. Isoforms may arise from normal allelic variation within a population and include mutations such as amino acid substitution, deletion, addition, truncation, or duplication. Also included within the term “functional equivalent” are variants generated at the level of transcription. Many kinases (including JAK2 and CDK2) have isoforms that arise from transcript variation. It is also known that FLT3 has an isoform that is the result of exon-skipping. Other functional equivalents include kinases having altered post-translational modification such as glycosylation.

Specific compounds of the invention include the following:

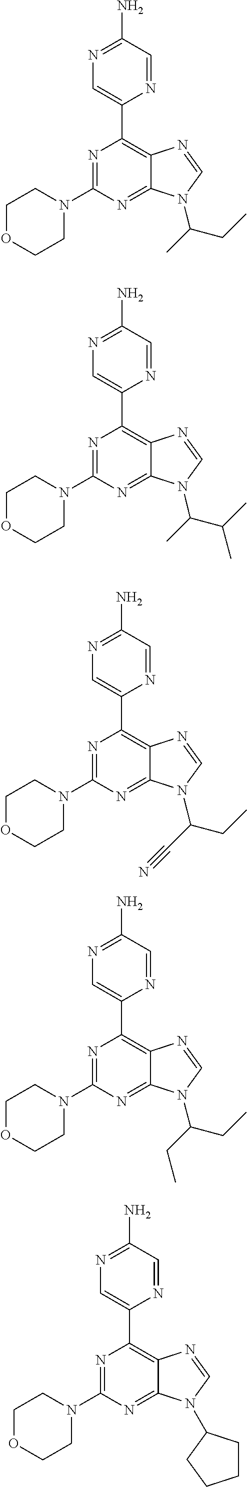

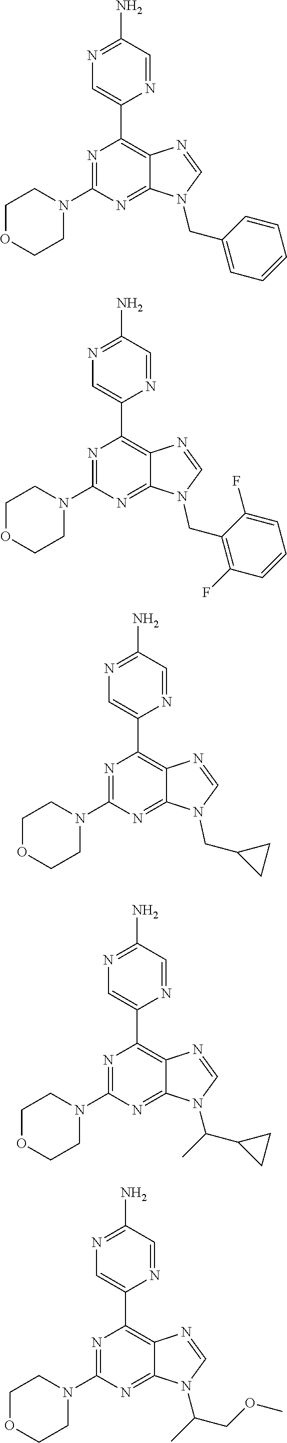

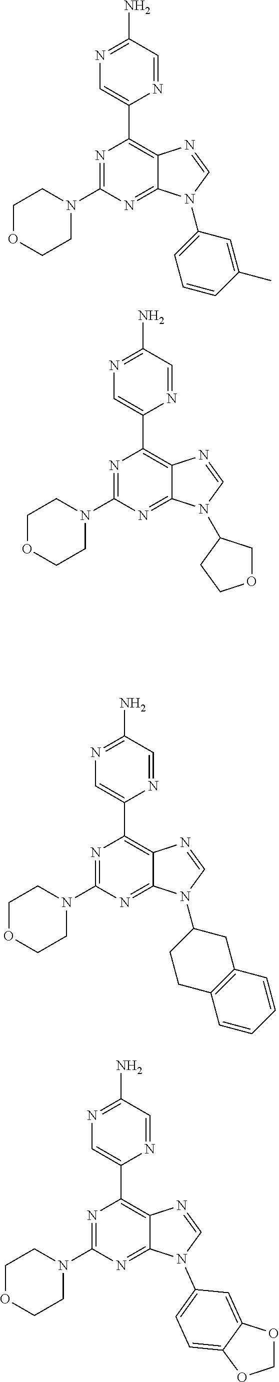

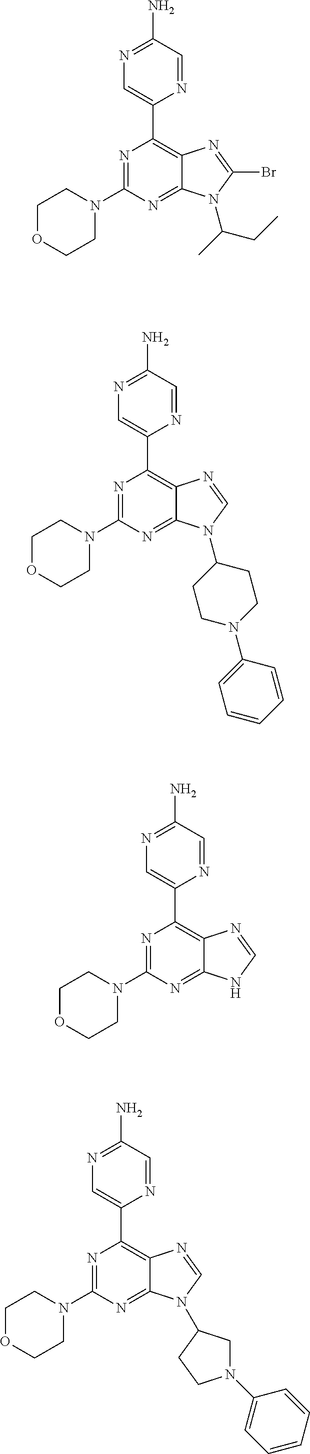

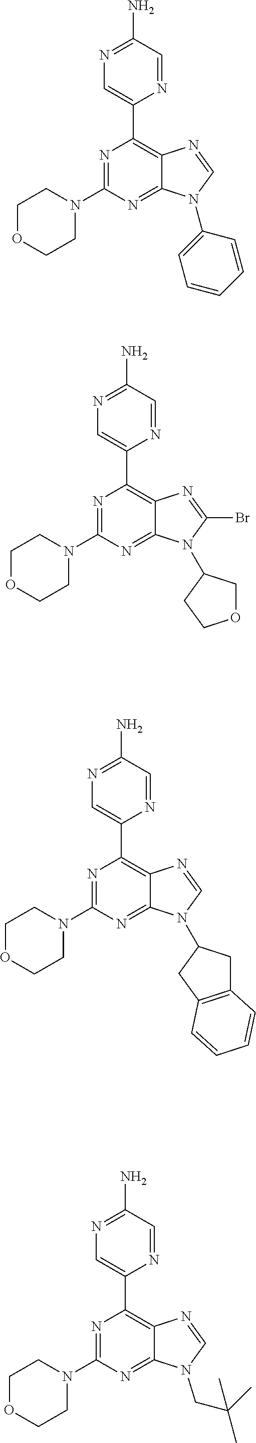

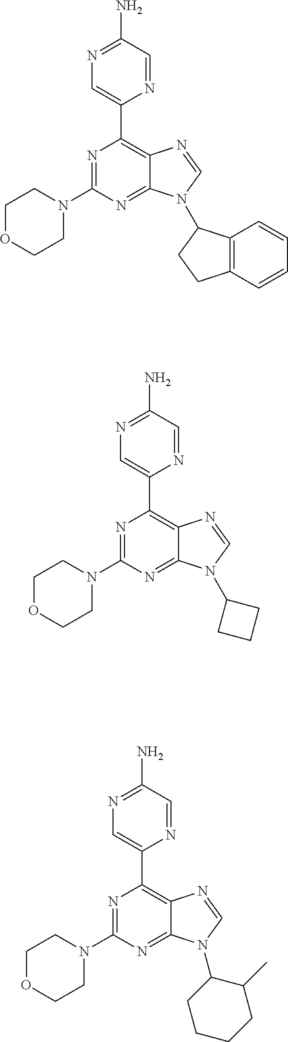

or a pharmaceutically acceptable salt or prodrug thereof.

The compounds have the ability to inhibit the activity of certain protein kinases. The ability to inhibit kinase activity may be a result of the compounds acting directly and solely on the kinase molecule to inhibit biological activity. However, it is understood that the compounds may also act at least partially on co-factors of the kinase in question that are involved in the phosphorylation process.

The compounds may have activity against PI3 protein kinases or a fragment or a complex or a functional equivalent thereof.

The compounds may have activity against certain serine/threonine kinases such as mTOR or a fragment or complex or functional equivalent thereof.

The inhibition of the protein kinase may be carried out in any of a number of well known ways in the art. For example if inhibition of the protein kinase in vitro is desired an appropriate amount of the compound may be added to a solution containing the kinase. In circumstances where it is desired to inhibit the activity of the kinase in a mammal the inhibition of the kinase typically involves administering the compound to a mammal containing the kinase.

Accordingly the compounds may find a multiple number of applications in which their ability to inhibit protein kinases of the type mentioned above can be utilised. For example the compounds may be used to inhibit serine/threonine protein kinases. The compounds may also be used in treating or preventing a condition in a mammal in which inhibition of a protein kinase and/or co-factor thereof prevents, inhibits or ameliorates a pathology or a symptomology of the condition.

It is anticipated that the compounds of the invention will be useful in treating various cancers including but not limited to bone cancers, brain and CNS tumours, breast cancers, colorectal cancers, endocrine cancers including adrenocortical carcinoma, pancreatic cancer, pituitary cancer, thyroid cancer, parathyroid cancer, thymus cancer, gastrointestinal cancers, Liver cancer, extra hepatic bile duct cancer, gastrointestinal carcinoid tumour, gall bladder cancer, genitourinary cancers, gynaecological cancers, head and neck cancers, leukemias, myelomas, hematological disorders, lung cancers, lymphomas, eye cancers, skin cancers, soft tissue sarcomas, adult soft tissue sarcoma, Kaposi's sarcoma, urinary system cancers.

Exemplary cancers that may be treated by compounds of this invention include Hematologic cancer such as myeloproliferative disorders (idiopathic myelofibrosis, polycythemia vera, essential thrombocythemia, chronic myeloid leukemia), myeloid metaplasia, chronic myelomonocytic leukemia, acute lymphocytic leukemia, acute erythroblastic leukemia, Hodgkin's and Non Hodgkin's disease, B-cell lymphoma, acute T-cell leukemia, myelodysplastic syndromes, plasma cell disorder, hairy cell leukemia, kaposi's sarcoma, lymphoma; gynaecologic cancer such as breast carcinoma, ovarian cancer, cervical cancer, vaginal and vulva cancer, endometrial hyperplasia; gastrointestinal tract cancer such as colorectal carcinoma, polyps, liver cancer, gastric cancer, pancreatic cancer, gall bladder cancer; urinary tract cancer such as prostate cancer, kidney and renal cancer; urinary bladder cancer, urethral cancer, penile cancer; skin cancer such as melanoma; brain tumour such as glioblastoma, neuroblastoma, astrocytoma, ependynoma, brain-stem gliomas, medulloblastoma, menigiomas, astrocytoma, oligodendroglioma; head and neck cancer such as nasopharyngeal carcinoma, laryngeal carcinoma; respiratory tract cancer such as lung carcinoma (NSCLC and SCLC), mesothelioma; eye disease such as retinoblastoma; musculo-skeleton diseases such as osteosarcoma, musculoskeleletal neoplasm; Squamous cell carcinoma and fibroid tumour.

The compounds may also be used the preparation of a medicament for treating a condition in an animal in which inhibition of a protein kinase can prevent, inhibit or ameliorate the pathology or symptomology of the condition. The compounds may also be used in the preparation of a medicament for the treatment or prevention of a kinase-related disorder.

Administration of compounds of formula (I) to humans can be by any of the accepted modes for enteral administration such as oral or rectal, or by parenteral administration such as subcutaneous, intramuscular, intravenous and intradermal routes. Injection can be bolus or via constant or intermittent infusion. The active compound is typically included in a pharmaceutically acceptable carrier or diluent and in an amount sufficient to deliver to the patient a therapeutically effective dose. In various embodiments the inhibitor compound may be selectively toxic or more toxic to rapidly proliferating cells, e.g. cancerous tumours, than to normal cells.

In using the compounds they can be administered in any form or mode which makes the compound bioavailable. One skilled in the art of preparing formulations can readily select the proper form and mode of administration depending upon the particular characteristics of the compound selected, the condition to be treated, the stage of the condition to be treated and other relevant circumstances. We refer the reader to Remingtons Pharmaceutical Sciences, 19^{th} edition, Mack Publishing Co. (1995) for further information.

The compounds can be administered alone or in the form of a pharmaceutical composition in combination with a pharmaceutically acceptable carrier, diluent or excipient. The compounds, while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. As such in some embodiments there is provided a pharmaceutical composition including a compound of formula (I) and a pharmaceutically acceptable carrier, diluent or excipient. The compositions are prepared in manners well known in the art.

The present teachings in other embodiments provide a pharmaceutical pack or kit comprising one or more containers filled with one or more of the ingredients of the pharmaceutical compositions. In such a pack or kit can be found a container having a unit dosage of the agent(s). The kits can include a composition comprising an effective agent either as concentrates (including lyophilized compositions), which can be diluted further prior to use or they can be provided at the concentration of use, where the vials may include one or more dosages. Conveniently, in the kits, single dosages can be provided in sterile vials so that the physician can employ the vials directly, where the vials will have the desired amount and concentration of agent(s). Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

The compounds may be used or administered in combination with one or more additional drug(s) for the treatment of the disorder/diseases mentioned. The components can be administered in the same formulation or in separate formulations. If administered in separate formulations the compounds may be administered sequentially or simultaneously with the other drug(s).

In addition to being able to be administered in combination with one or more additional drugs, the compounds may be used in a combination therapy. When this is done the compounds are typically administered in combination with each other. Thus one or more of the compounds may be administered either simultaneously (as a combined preparation) or sequentially in order to achieve a desired effect. This is especially desirable where the therapeutic profile of each compound is different such that the combined effect of the two drugs provides an improved therapeutic result.

Pharmaceutical compositions of the present teaching for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminium metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, patches, sprays, ointments and inhalants. The active compound is mixed under sterile conditions with a pharmaceutically acceptable carrier and any needed preservatives, buffers, or propellants which may be required.

The amount of compound administered will preferably treat and reduce or alleviate the condition. A therapeutically effective amount can be readily determined by an attending diagnostician by the use of conventional techniques and by observing results obtained under analogous circumstances. In determining the therapeutically effective amount a number of factors are to be considered including but not limited to, the species of animal, its size, age and general health, the specific condition involved, the severity of the condition, the response of the patient to treatment, the particular compound administered, the mode of administration, the bioavailability of the preparation administered, the dose regime selected, the use of other medications and other relevant circumstances.

A preferred dosage will be a range from about 0.01 to 300 mg per kilogram of body weight per day. A more preferred dosage will be in the range from 0.1 to 100 mg per kilogram of body weight per day, more preferably from 0.2 to 80 mg per kilogram of body weight per day, even more preferably 0.2 to 50 mg per kilogram of body weight per day. A suitable dose can be administered in multiple sub-doses per day.

### Synthesis of Compounds of the Invention

The agents of the various embodiments may be prepared using the reaction routes and synthesis schemes as described below, employing the techniques available in the art using starting materials that are readily available. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T. W. Greene's Protective Groups in Organic Synthesis, 3^{rd} Edition, John Wiley & Sons, 1991. Alternatively, other reactions disclosed herein or known in the art will be recognized as having applicability for preparing other compounds of the various embodiments.

Reagents useful for synthesizing compounds may be obtained or prepared according to techniques known in the art.

### General Synthetic Scheme

A wide range of trisubstituted purines can be prepared in a straightforward three step procedure starting from 2,6-dichloropurine which is commercially available from a number of sources or may be prepared from purine itself using, for example, phosphorylchloride. The general representative procedure is shown in scheme 1.

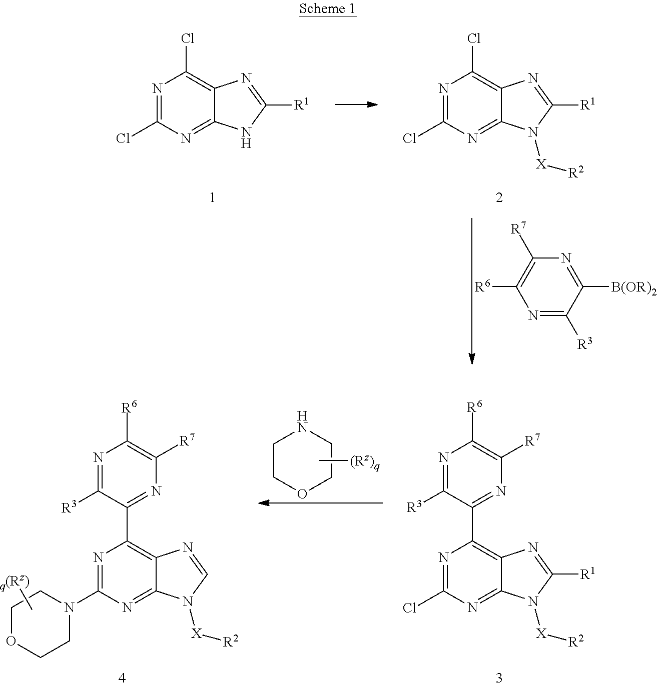

As shown, initial reaction of 2,6-dichloropurine with an alkyl halide results in alkylation predominately at the 9 position (Tetrahedron Letters 1998, 36, 11, 1945). A typical procedure uses an alkyl bromide in the presence of a suitable base such as potassium carbonate. Alternatively, an alcohol may be reacted with the 2,6-dichloropurine in the presence of a phosphine and an activating agent, such as diethylazodicarboxylate, so as to effect a similar alkylation. N-arylation may also be carried out at the 9 position of the dichloropurine. Copper catalysed couplings of this type have been described by Gundersen et al. in Tetrahedron Letters 2003, 44, 3359-3362. Subsequent palladium catalysed coupling of 2 with a suitable aryl boronic acid or ester then delivers intermediate 3 (Collect. Czech. Chem. Commun. 2002, 67, 325). Addition of morpholine can then be carried out at elevated temperature, in a suitable solvent such as DMA, DMF or THF, to give the desired trisubstituted purine. In cases where a substituted morpholine group is being added the reaction has been shown to be facilitated by the use of microwave irradiation.

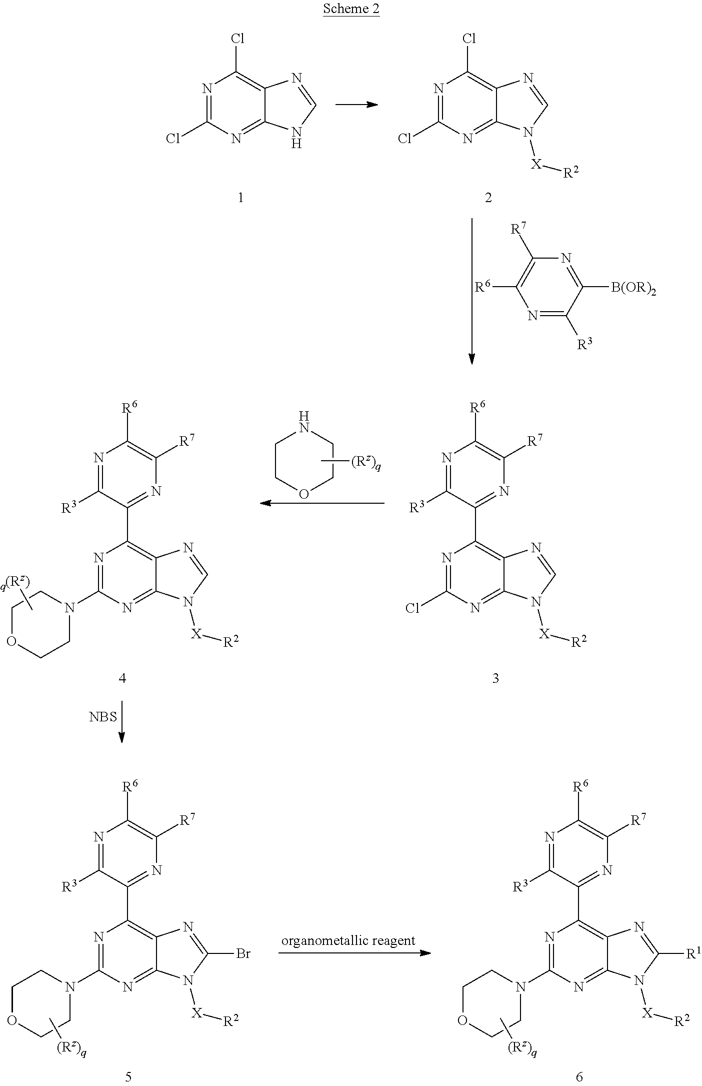

The R¹ substituent may be varied either by using an 8-substituted dichloropurine as starting material (Scheme 1) or can be introduced later in the synthetic sequence (Scheme 2). For example chemistry may be carried out on the 8-position after completion of the sequence illustrated in scheme 1 above. For example, the 8-position of 4 may be brominated to give 5. The bromide may then be displaced by, for example, an organometallic agent, such as an organozinc, to install R¹ as in 6.

### EXAMPLES

In the examples described below, unless otherwise indicated, all temperatures in the following description are in degrees Celsius and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated. Tetrahydrofuran (THF) and N,N-dimethylformamide (DMF) were purchased from Aldrich in SureSeal bottles and used as received. All solvents were purified by using standard methods in the art, unless otherwise indicated.

The reactions set forth below were performed under a positive pressure of nitrogen, argon or with a drying tube, at ambient temperature (unless otherwise stated), in anhydrous solvents, and the reaction flasks are fitted with rubber septa for the introduction of substrates and reagents via syringe. Glassware was oven-dried and/or heat-dried. Analytical thin-layer chromatography was performed on glass-backed silica gel 60 F 254 plates (E Merck (0.25 mm)) and eluted with the appropriate solvent ratios (v/v). The reactions were assayed by TLC and terminated as judged by the consumption of starting material.

The TLC plates were visualized by UV absorption or with a p-anisaldehyde spray reagent or a phosphomolybdic acid reagent (Aldrich Chemical, 20 wt % in ethanol) which was activated with heat, or by staining in an iodine chamber. Work-ups were typically done by doubling the reaction volume with the reaction solvent or extraction solvent and then washing with the indicated aqueous solutions using 25% by volume of the extraction volume (unless otherwise indicated). Product solutions were dried over anhydrous sodium sulfate prior to filtration, and evaporation of the solvents was under reduced pressure on a rotary evaporator and noted as solvents removed in vacuo. Flash column chromatography [Still et al, J. Org. Chem., 43, 2923 (1978)] was conducted using E Merck-grade flash silica gel (47-61 mm) and a silica gel:crude material ratio of about 20:1 to 50:1, unless otherwise stated. Hydrogenolysis was done at the pressure indicated or at ambient pressure.

¹H NMR spectra were recorded on a Bruker instrument operating at 400 MHz, and ¹³C-NMR spectra was recorded operating at 100 MHz. NMR spectra were obtained as CDCl₃ solutions (reported in ppm), using chloroform as the reference standard (7.27 ppm and 77.00 ppm) or CD₃OD (3.4 and 4.8 ppm and 49.3 ppm), or an internal tetramethylsilane standard (0.00 ppm) when appropriate. Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Mass spectra were obtained using LC/MS either in ESI or APCI. All melting points are uncorrected.

All final products had greater than 90% purity (by HPLC at wavelengths of 220 nm and 254 nm).

The following examples are intended to illustrate the embodiments disclosed and are not to be construed as being limitations thereto. Additional compounds, other than those described below, may be prepared using the following described reaction scheme or appropriate variations or modifications thereof.

Scheme 3 depicts three variations on the three step procedure in which different conditions are used in the first step so as to introduce diverse substituents at the 9-position of the purine scaffold. In principle, however, a skilled addressee could modify the general reaction scheme shown in scheme one where the nitrogen moiety at the 9 position of the purine may be reacted with a moiety containing a suitable leaving group (such as a halide) in a reaction whereby the nitrogen displaces the leaving group to form the compound in which the nitrogen at the 9 position is then functionalised with the moiety. Suitable leaving groups for use in reactions of this type which can be displaced by nitrogen in such reactions are known in the art and in general the synthesis of moieties containing leaving groups of this type for use in these types of reactions are also well known to a skilled worker in the field.

As shown in Scheme 3 the three simplest routes to the compounds of the invention involve reaction of the dichloropurine with either an arylalkyl halide (such as benzyl halide) or a heteroarylalkyl halide to introduce an aryl or heteroaryl substituted methyl group at the 9 position, an alcohol (to introduce a di-substituted methyl group at the 9 position) or an aryl or heteroaryl boronic acid (to introduce an aryl or heteroaryl group directly.

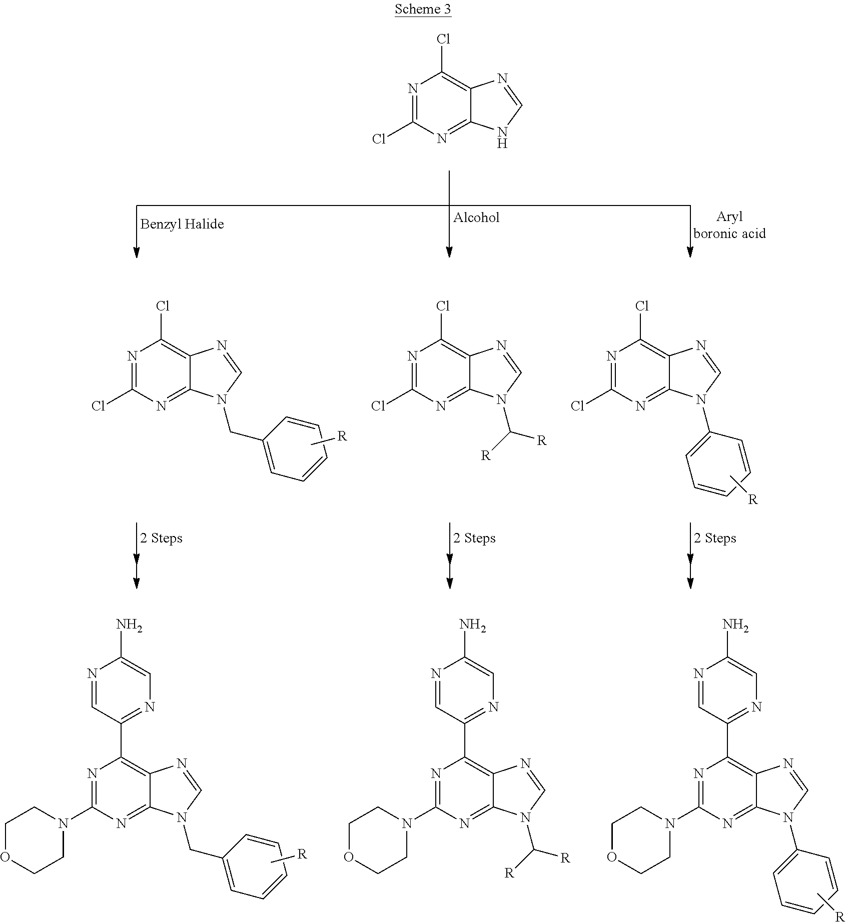

### Example 1

### Compound 1

### Synthesis of 9-sec-Butyl-2,6-dichloro-9H-purine

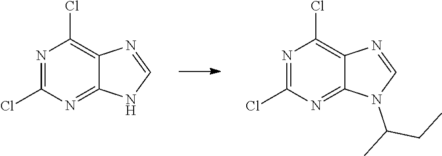

2,6-Dichloropurine (5.3 mmol), 2-butanol (9.01 mmol) and triphenylphosphine (7.95 mmol) were dissolved in 40 ml anhydrous tetrahydrofuran, to which was added drop-wise diisoproplyazidodicarboxylate (7.95 mmol) at room temperature over a period of 30 minutes. The reaction mixture was stirred at room temperature for 24 h. Conversion was monitored by TLC or LC/MS. The reaction mixture was poured into a beaker containing ice-cold water. Extraction of the aqueous layer, using 3×100 ml portions of ethyl acetate, afforded the crude product. This was purified on the silica gel column (10-80% ethyl acetate in petroleum ether, gradient elution) to give the desired compound in a yield of 50%.

### Synthesis of 5-(9-sec-Butyl-2-morpholin-4-yl-9H-purin-6-yl)-pyrazin-2-ylamine

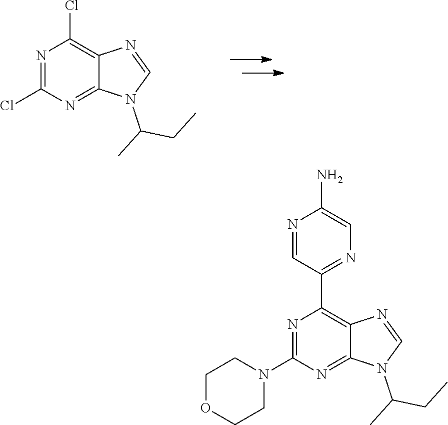

A solution of 9-sec-butyl-2,6-dichloro-9H-purine (0.204 mmol), 5-aminopyrazine-2-boronic acid pinacol ester (0.204 mmol) and 1,1′-bis(diphenylphosphino) ferrocene palladium (II) chloride, complexed with dichloromethane, (0.02 mmol) were taken up in a mixture of peroxide free dioxane (40 ml) and added 2M aqueous solution of sodium carbonate (0.816 mmol). The reaction mixture was degassed and purged with nitrogen. This reaction mixture was then stirred on an oil bath maintained at 80° C. for 3 h. Conversion was monitored by LC/MS for the disappearance of the starting purine. The reaction mixture was cooled to room temperature and the solvents removed under reduced pressure. The residue was taken up in ethyl acetate and water. The organic phase was separated and the aqueous layer further extracted with 3×100 ml portions of ethyl acetate. The organics were dried over sodium sulfate and the solvents removed under vacuum to give 5-(9-sec-butyl-2-chloro-9H-purin-6-yl)-pyrazin-2-ylamine. This was taken up in dimethyl acetamide (5 ml) and morpholine (0.5 mmol) was added to the solution. The reaction mixture was then heated on an oil bath maintained at 94° C. for 12 h. Conversion was monitored by LC-MS. The crude material was directly loaded onto a preparative HPLC column and purified by chromatography to give compound 1 (3.54 mg). ¹H NMR, DMSO-d6: δ 92 (s, 1H), 9.1 (s, 1H), 8.1 (s, 1H), 7.4 (bs, 2H), 4.6 (m, 1H), 3.85 (m, 4H), 3.74 (m, 4H), 2.09 (m, 1H), 1.99 (m, 1H), 1.6 (d, 3H), 0.85 (t, 3H). m/z: 355.23 [MH]⁺.

### Example 2

### Compound 7

### Synthesis of 2,6-Dichloro-9-(2,6-difluoro-benzyl)-9H-purine

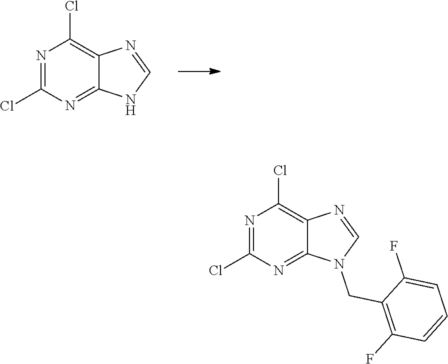

To a stirred solution of 2,6-dichloropurine (5.3 mmol) in 10 ml anhydrous DMSO at room temperature was added anhydrous potassium carbonate (6.34 mmol) and 2,6-difluorobenzylbromide (6.34 mmol). The reaction mixture was maintained at this temperature for 20 h. The reaction can be monitored using either TLC or LC/MS. The reaction mixture was poured in to a beaker containing ice-cold water. The aqueous layer was acidified to pH 5-6. Extraction of the aqueous layer, using 3×75 ml portions of ethyl acetate, afforded the crude product. This was purified on the silica gel column (10-70% ethyl acetate in petroleum ether, step-gradient), to give the desired compound in a yield of 61%.

### Synthesis of 5-[9-(2,6-Difluoro-benzyl)-2-morpholin-4-yl-9H-purin-6-yl]-pyrazin-2-ylamine

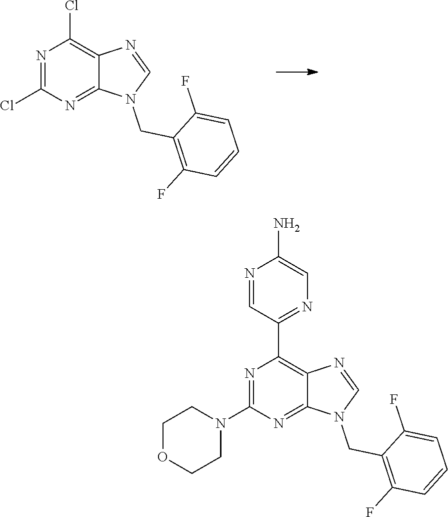

A solution of 2,6-dichloro-9-(2,6-difluoro-benzyl)-9H-purine (0.159 mmol), 5-aminopyrazine-2-boronic acid pinacol ester (0.159 mmol) and 1,1′-bis(diphenylphosphino) ferrocene palladium (II) chloride, complexed with dichloromethane, (0.015 mmol) was taken up in a mixture of peroxide free dioxane (40 ml) and 2M aqueous solution of sodium carbonate (0.760 mmol) added. The reaction mixture was degassed and purged with nitrogen. This reaction mixture was then stirred on an oil bath maintained at 65° C. for 3 h. The reaction was monitored by LC/MS for the disappearance of the starting purine. The reaction mixture was cooled to room temperature and the solvents removed under reduced pressure. The residue was taken up in ethyl acetate and water. The organic phase was separated and the aqueous layer further extracted with 3×100 ml portions of ethyl acetate. The combined ethyl acetate layers were washed once with brine solution (25 ml). The organics were dried over sodium sulfate and the solvents removed under vacuum to give 5-[2-chloro-9-(2,6-difluoro-benzyl)-9H-purin-6-yl]-pyrazin-2-ylamine. This material was taken up in dimethyl acetamide (5 ml) and morpholine (0.5 mmol) added. The reaction mixture was heated on an oil bath maintained at 94° C. for 12 h. Conversion was monitored by LC-MS. The crude material was directly loaded onto a preparative HPLC column and purified by chromatography to give compound 7 (2.3 mg). ¹H NMR, DMSO-d6: δ 9.21 (s, 1H), 8.68 (br.s, 1H), 8.07 (s, 1H), 7.48 (m, 2H), 7.19-7.14 (m, 3H), 5.48 (s, 2H), 3.78-3.77 (m, 4H), 3.69-3.67 (m, 4H). m/z: 425.16 [MH]⁺.

### Example 3

### Compound 11

### Synthesis of 2,6-Dichloro-9-m-tolyl-9H-purine

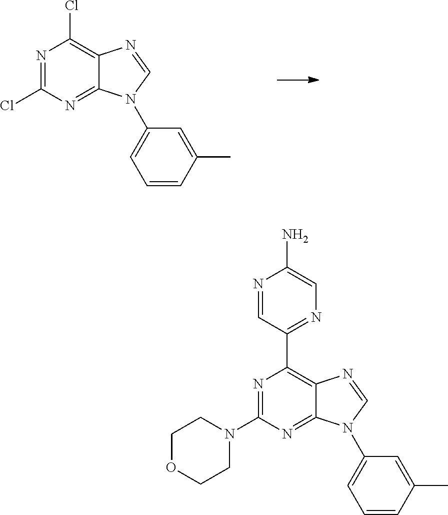

2,6-Dichloropurine (1.3 mmol), m-tolyl boronic acid (4.0 mmol), anhydrous cupric acetate (1.32 mmol), 4 Å molecular sieves (1 g) and [1,10]-phenanthroline (2.64 mmol) in 25 ml of anhydrous dichloromethane were stirred at room temperature in a round bottomed flask. The reaction mixture was stirred at room temperature and monitored by TLC, LC-MS. Reaction was complete after 24 h. The molecular sieves and inorganic material were removed by filtration through a celite bed. The bed was thoroughly washed with methanol. The combined organics were purified by flash chromatography to yield 50% of the desired compound as a solid. m/z: 279.02 [MH]+.

### Synthesis of 5-(2-Morpholin-4-yl-9-m-tolyl-9H-purin-6-yl)-pyrazin-2-ylamine

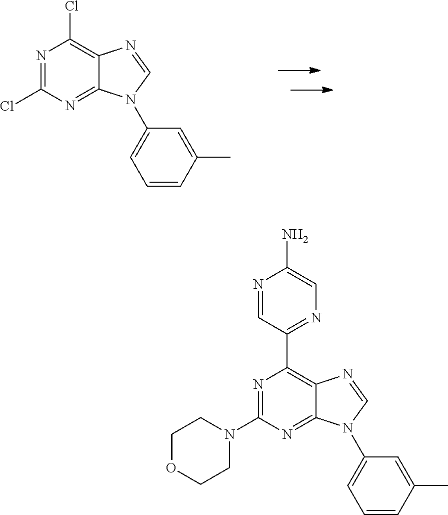

2,6-Dichloro-9-m-tolyl-9H-purine (0.182 mmol), 5-(4,4,5,5-tetramethyl-[1,3,2] dioxaborolan-2-ylamine (0.182 mmol) and 1,1′-bis(diphenylphosphino) ferrocene palladium (II) chloride, complexed with dichloromethane, (0.018 mmol) were taken up in a mixture of peroxide free dioxane (40 ml) and added 2M aqueous solution of sodium carbonate (0.730 mmol). The reaction mix was degassed and purged with nitrogen. This reaction mix was then stirred on an oil bath maintained at 40° C. for 2 h. The reaction was monitored by LC/MS for the disappearance of the starting purine. The reaction mixture was cooled to room temperature and the solvents removed under reduced pressure. The residue was taken up in ethyl acetate and water. The organic phase was separated and the aqueous layer further extracted with 3×100 ml portions of ethyl acetate. The organics were dried over sodium sulfate and the solvents removed under vacuum to give 5-(2-chloro-9-m-tolyl-9H-purin-6-yl)-pyrazin-2-ylamine. This crude material was taken directly to the next step without further purification. After dissolution in dimethyl acetamide (4 ml) morpholine (0.4 mmol) was added. The reaction mixture was heated on an oil bath maintained at 94° C. for 12 h. The reaction was monitored by LC-MS. The crude material was directly loaded onto a preparative HPLC column and purified by chromatography to give compound 11 (3.13 mg). ¹H-NMR, (DMSO-d6): δ 9.32 (s, 1H), 8.77 (br.s, 1H), 8.11 (s, 1H), 7.75 (s, 1H), 7.29 (t, 1H), 7.16 (d, 1H), 7.15 (br.s, 2H), 3.81-3.79 (m, 4H), 3.73-3.71 (m, 4H), 2.44 (s, 3H). m/z: 389.14 [MH]⁺.

### Example 4

### Compound 15

### Synthesis of 5-(8-Bromo-9-sec-butyl-2-morpholin-4-yl-9H-purin-6-yl)-pyrazin-2-ylamine

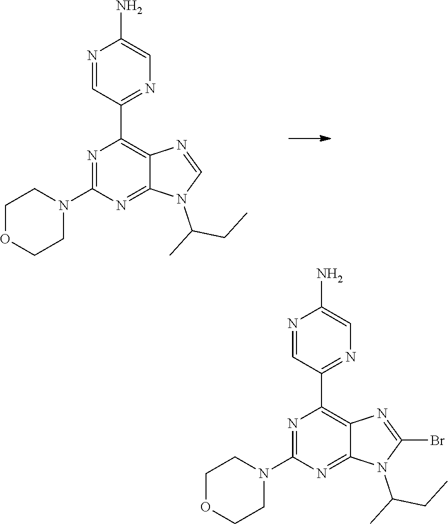

To a solution of 5-(9-sec-butyl-2-morpholin-4-yl-9H-purin-6-yl)-pyrazin-2-ylamine 1 (100 mg, 0.29 mmol) in 15 ml of chloroform, was slowly added N-bromosuccinimide (60 mg, 0.34 mmol) at a temperature of 5° C. The reaction was continued for 2 hours at this temperature. After simple work-up, the product 5-(8-bromo-9-sec-butyl-2-morpholin-4-yl-9H-purin-6-yl)-pyrazin-2-ylamine 15 was purified by flash column chromatography (solvent system: 50% ethyl acetate in hexane) and isolated in a yield of 24% (30 mg). ¹H-NMR, (MeOD): δ 9.12 (s, 1H), 9.00 (s, 1H), 4.67 (m, 1H), 3.84 (m, 4H), 3.70 (m, 4H), 2.08 (m, 1H), 1.95 (m, 1H), 1.60 (d, 3H), 0.83 (t, 3H). m/z: 433.06, 435.06 [MH]⁺.

The compounds outlined in Table 1 were synthesized following the procedures outlined above or variations thereof.

TABLE 1
Synthesised compounds

| No | Structure | ¹H NMR | m/z |
|---|---|---|---|
| 1 | 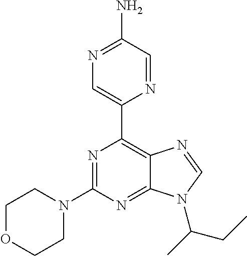 | (DMSO-d6) δ 9.2 (s, 1H), 9.1 (s, 1H), 8.1 (s, 1H), 7.4 (bs, 2H), 4.6 (m, 1H), 3.85 (m, 4H), 3.74 (m, 4H), 2.09 (m, 1H), 1.99 (m, 1H), 1.6 (d, 3H), 0.85 (t, 3H) | 355.23 |
| 2 | 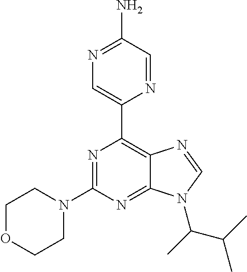 | (CDCl₃) δ 9.10 (s, 1H), 8.84 (s, 1H), 8.40 (s, 1H), 5.78 (bs, 2H), 4.54 (m, 1H), 3.98 (m, 4H), 3.88 (m, 4H), 3.72 (m, 1H), 1.66 (d, 3H), 1.07 (d, 3H), 0.94 (d, 3H) | 369.26 |
| 3 | 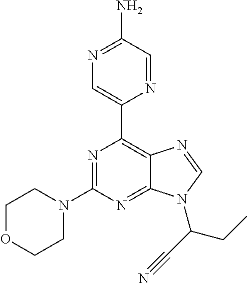 | (DMSO-d6) δ 9.26 (s, 1H), 8.41 (s, 1H), 8.09 (s1H), 7.2 (bs, 2H), 5.82 (m, 1H), 3.82 (m, 4H), 3.74 (m, 4H), 2.4 (m, 2H), 0.97 (t, 3H) | 366.18 |
| 4 | 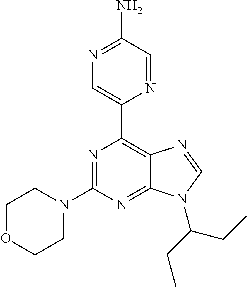 | (DMSO-d6) δ 9.20 (s, 1H), 9.0 (s, 1H), 8.10 (s, 1H), 7.30 (bs, 2H), 4.40 (m, 1H), 3.81 (m, 4H), 3.73 (m, 4H), 2.09 (m, 2H), 1.99 (m, 2H), 0.85 (t, 6H) | 369.21 |
| 5 | 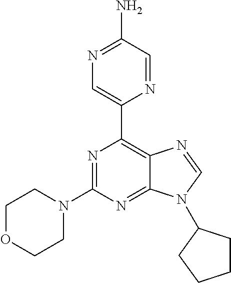 | (CD₃OD) δ 9.22 (s, 1H), 8.96 (s, 1H), 8.06 (s, 1H), 4.96 (m, 1H), 3.86 (m, 4H), 3.71 (m, 4H), 2.28 (m, 2H), 2.10 (m, 2H), 1.92 (m, 2H), 1.75 (m, 2H) | 367.17 |
| 6 | 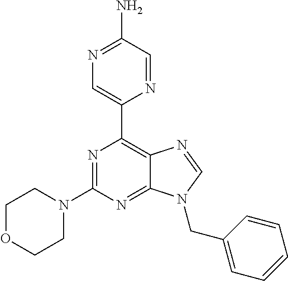 | (DMSO-d6) δ 9.22 (s, 1H), 8.82 (br. s, 1H), 8.09 (s, 1H), 7.44 (d, 2H), 7.40-7.31 (m, 3H), 7.23 (br. s, 2H), 5.42 (s, 2H), 3.83- 3.77 (m, 4H), 3.73-3.71 (m, 4H) | 389.14 |
| 7 | 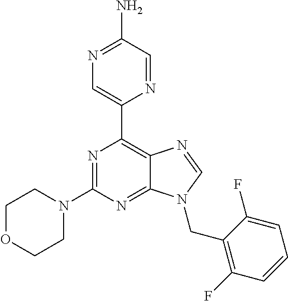 | (DMSO-d6) δ 9.21 (s, 1H), 8.68 (br. s, 1H), 8.07 (s, 1H), 7.48 (m, 2H), 7.19-7.14 (m, 3H), 5.48 (s, 2H), 3.78-3.77 (m, 4H), 3.69-3.67 (m, 4H) | 425.16 |
| 8 | 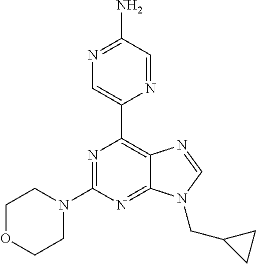 | (DMSO-d6) δ 9.22 (s, 1H), 8.91 (br. s, 1H), 8.11 (s, 1H), 7.29 (br. s, 2H), 4.08 (d, 2H), 3.86- 3.84 (m, 4H), 3.78-3.72 (m, 4H), 1.39 (m, 1H), 0.60-0.45 (m, 4H) | 353.18 |
| 9 | 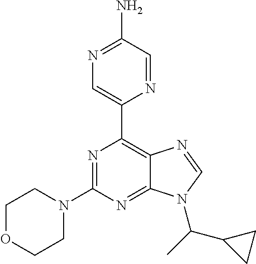 | (DMSO-d6) δ 9.19 (s, 1H), 9.15 (br. s, 1H), 8.12 (s, 1H), 7.35 (br. s, 2H), 3.99 (m, 1H), 3.84- 3.79 (m, 4H), 3.78-3.72 (m, 4H), 1.68 (d, 3H), 1.57 (m, 1H), 0.72- 0.68 (m, 1H), 0.55-0.35 (m, 3H) | 367.17 |
| 10 | 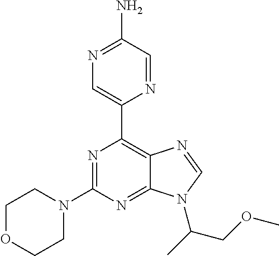 | (DMSO-d6) δ 9.22 (s, 1H), 8.85 (br. s, 1H), 8.11 (s, 1H), 7.27 (br. s, 2H), 4.91 (m, 1H), 3.89 (dd, 1H), 3.91-3.87 (m, 4H), 3.83-3.66 (m, 6H), 3.26 (s, 3H), 1.55 (d, 3H) | 371.17 |
| 11 | 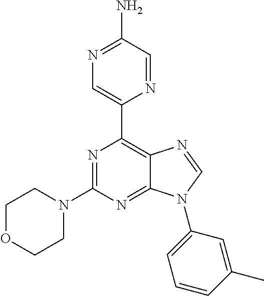 | (DMSO-d6) δ 9.32 (s, 1H), 8.77 (br. s, 1H), 8.11 (s, 1H), 7.75 (s, 1H), 7.29 (t, 1H), 7.16 (d, 1H), 7.15 (br. s, 2H), 3.81- 3.79 (m, 4H), 3.73-3.71 (m, 4H), 2.44 (s, 3H) | 389.14 |
| 12 | 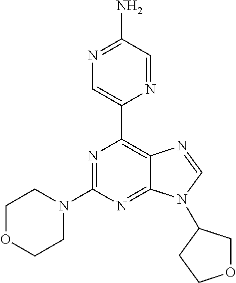 | (DMSO-d6) δ 9.08 (d, 1H), 8.93 (s, 1H), 8.03 (d, 1H, d), 7.30 (br. s, 2H), 5.24-5.19 (m, 1H), 4.11-3.91 (m, 3H), 3.83-3.64 (m, 9H), 2.36-2.30 (m, 2H) | 369.15 |
| 13 | 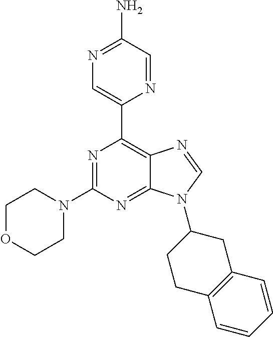 | (DMSO-d6) δ 9.50 (s, 1H), 9.10 (s, 1H), 8.30 (s, 1H), 7.50 (br. s, 2H), 7.20 (m, 4H), 5.00 (m, 1H), 3.80 (m, 4H), 3.60 (m, 4H), 3.50 (m, 1H), 3.20 (m, 1H), 3.00 (m, 2H), 2.40 (m, 2H) | 429.25 |
| 14 | 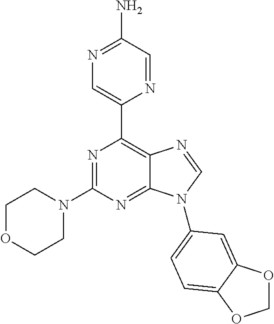 | (DMSO-d6) δ 9.30 (s, 1H), 8.60 (s, 1H), 8.10 (s, 1H), 7.50 (s, 1H), 7.30 (d, 1H), 7.10 (m, 3H), 6.10 (s, 2H), 3.78 (m, 4H), 3.72 (m, 4H) | 419.16 |
| 15 | 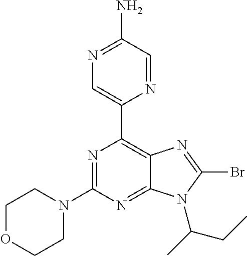 | (MeOD) δ 9.12 (s, 1H), 9.00 (s, 1H), 4.67 (m, 1H), 3.84 (m, 4H), 3.70 (m, 4H), 2.08 (m, 1H), 1.95 (m, 1H), 1.60 (d, 3H), 0.83 (t, 3H) | 433.06, 435.06 |
| 16 | 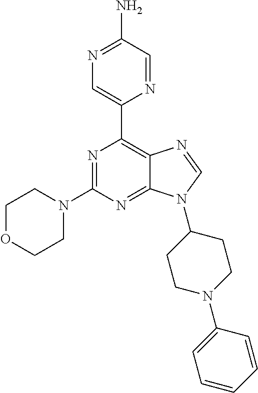 | (DMSO-d6) δ 9.18 (s, 1H), 9.13 (s, 1H), 8.09 (s, 1H), 7.39 (br. s, 2H), 7.25 (t, 2H), 7.03 (d, 2H), 6.80 (t, 1H), 4.77-4.71 (m, 1H), 3.95 (d, 2H), 3.68-3.59 (m, 8H), 2.97 (t, 2H), 2.44-2.33 (m, 2H), 2.10 (d, 2H) | 458.20 |
| 17 | 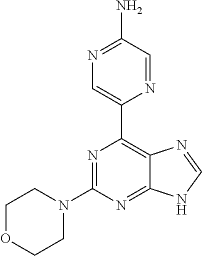 | (DMSO-d6) δ 9.04 (d, 1 H), 8.81 (s, 1 H), 8.09 (d, 1H), 7.29 (br. s, 2H), 3.79-3.70 (m, 8H) | 299.08 |
| 18 | 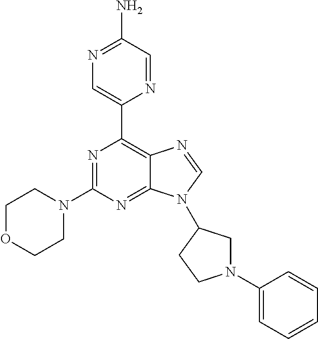 | (DMSO-d6) δ 9.15 (d, 1H), 9.05 (s, 1H), 8.09 (d, 1H), 7.36 (br. s, 2H), 7.21 (t, 2H), 6.69-6.62 (m, 3H), 5.43-5.37 (m, 1H), 3.86- 3.61 (m, 11H), 3.47-3.42 (m, 1H), 2.69-2.59 (m, 2H) | 444.17 |
| 19 | 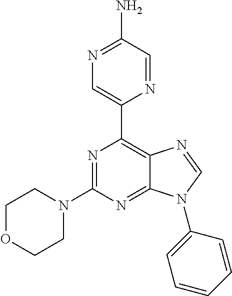 | (DMSO-d6, ppm) δ 9.16 (s, 1H), 8.3 (s, 1H), 8.13 (s, 1H), 7.7 (d, 2H), 7.5 (t, 2H), 7.3 (m, 1H), 6.0 (br. s, 2H), 4.0 (m, 4H), 3.8 (m, 4H) | 375.15 |
| 20 | 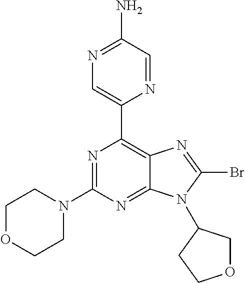 | (CDCl₃) δ 9.27 (s, 1H), 7.99 (s, 1H), 5.42 (s, 2H), 5.27-5.22 (m, 1), 4.24-4.18 (m, 1H), 4.14- 3.98 (m, 3H), 3.94-3.82 (m, 8H), 2.62-2.52 (m, 1H), 2.27- 2.21 (m, 1H) | 447.05, 449.05 |
| 21 | 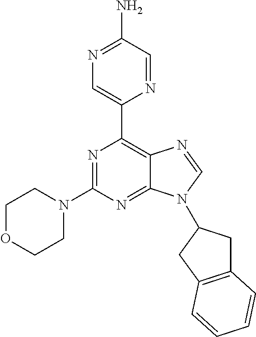 | (DMSO-d6) δ 9.18 (s, 1H), 8.8 (s, 1H), 8.1 (s, 1H), 7.2-7.4 (m, 6H), 5.5 (m, 1H), 3.7-3.6 (m, 8H), 3.5 (m, 4H) | 415.20 |
| 22 | 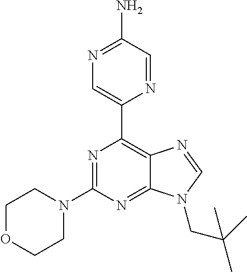 | (DMSO-d6) δ 9.20 (s, 1H), 8.80 (s, 1H), 8.10 (s, 1H), 7.30 (br. s, 2H), 4.00 (s, 2H), 3.80-3.70 (m, 8H), 0.98 (s, 9H) | 369.21 |
| 23 | 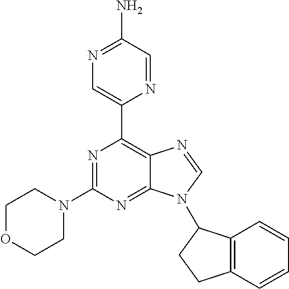 | (DMSO-d6) δ 9.20 (s, 1H), 8.60 (s, 1H), 8.10 (s, 1H), 7.50-7.20 (m, 6H), 6.20 (s, 1H), 3.60-3.80 (m, 8H), 3.40 (m, 1H), 3.10 (m, 1H), 2.60 (m, 1H), 2.40 (m, 1H) | 415.20 |
| 24 | 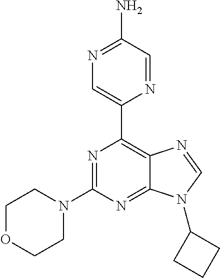 | (DMSO-d6) δ 9.19-9.13 (m, 2H), 8.11 (s, 1H), 7.33 (br. s, 2H), 5.07 (q, 1H), 3.85-3.84 (m, 4H), 3.78-372 (m, 4H), 2.75-2.49 (m, 4H), 1.96-1.90 (m, 2H) | 353.24 |
| 25 | 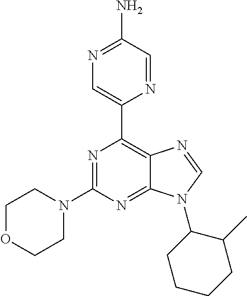 | (DMSO-d6) δ 9.20 (s, 1H), 8.94 (m 1H), 8.11 (s, 1H), 7.22 (br. s, 2H), 5.02-4.95 (m, 1H), 3.90- 3.65 (m, 4H), 2.35 (m, 1H), 1.95- 1.45 (m, 8H), 0.68 (m, 3H) | 395.27 |

### Biological Testing

mTOR Assay

Full-length mTOR kinase and His-tagged 4eBP1 were produced in-house. [γ³³P]-ATP was purchased from Amersham (GE Healthcare). All chemicals, unless otherwise stated, were from Sigma-Aldrich.

Phosphorylation assays were initially performed in a final volume of 20 μL in 384-well polypropylene plate (Greiner). Compounds were typically tested over the range from 100 μM to 0.006 μM, in 8 step dilutions, in duplicate. 10 μL/well of 2× Enzyme-Substrate solution (1.5 μg/mL mTOR, 40 μg/mL 4eBP1 in 1× assay buffer: 10 mM Hepes pH 7.5, 50 mM NaCl and 10 mM MnCl₂) were first added to the sample plate containing 1 μL/well of test compound in neat DMSO. The reaction was initiated by adding 10 μL/well of 20 μM ATP solution (final assay concentration 10 μM ATP and 0.4 μCi/well of [γ³³P]-ATP). After 1 hour incubation at room temperature, the reaction was terminated with 40 μL/well of 20 mM EDTA/1 mM ATP solution.

50 μL/well of the stopped reaction mix was then transferred to 384-well MultiScreenHTS-PH filter plate (Millipore) pre-added with 50 μL/well of 1% phosphoric acid. The plate was washed 4 times with 120 μL/well of 0.5% phosphoric acid via vacuum filtration. Finally, 10 μL/well of Optiphase™ SuperMix liquid scintillation cocktail (Perkin Elmer) was added. After minimum 1 hour of incubation, counting was performed in a Wallac MicroBeta TriLux scintillation counter using coincidence counting mode with crosstalk correction. IC₅₀ is defined as the concentration of compound required for 50% inhibition of kinase enzyme activity. IC50 data are shown in Table 2 below.

### PI3K Assay

Recombinant PI3K p110α/p85 was prepared in-house. Phosphatidylinositol (PtdIns), phosphotidylserine (PtdSer) and all other unspecified chemicals were purchased from Sigma-Aldrich. [γ33P]ATP and Optiphase scintillant were obtained from Perkin Elmer.

Assays were performed in a final assay volume of 25 μL in 384-well Maxisorp plates (Nunc). Compounds were tested at 8 concentrations in 3-fold serial dilution, generally starting from 10 μM. Maxisorp plates were coated with 20 μL/well of a 1:1 mixture of PtdIns and PtdSer [0.1 mg/mL each dissolved in chloroform:ethanol (3:7)] and left overnight in a fume hood at room temperature to dry.

The enzyme reaction was created by pipetting 5 μL/well of compound (in 2.5% DMSO), 10 μL/well of enzyme (0.5 μg/mL p110α+1 μg/mL p85), and 10 μL/well of 5 μM ATP with 5 μCi/mL [γ³³P]ATP in assay buffer (final concentrations: 0.2 μg/mL p110α, 2 μM ATP, 0.05 μCi/well [γ³³P]ATP in 1× assay buffer: 100 mM Tris-HCl pH 7.0, 200 mM NaCl, 8 mM MgCl₂). The reaction was incubated for 1 hour at RT and terminated with 30 μL/well of 50 mM EDTA solution. The plate was then washed twice with TBS, dried, and added with 30 μL/well of scintillant before it was counted in a MicroBeta Trilux. IC₅₀ is defined as the concentration of compound required for 50% inhibition of kinase enzyme activity. IC₅₀ data are shown in Table 2 below.

TABLE 2
In vitro mTOR and PI3K inhibition activity assay IC₅₀ data

| Com- | | | Com- | | |
|---|---|---|---|---|---|
| pound | IC₅₀ | IC₅₀ | pound | IC₅₀ | IC₅₀ |
| Number | (mTOR)* | (PI3Kα)* | Number | (mTOR)* | (PI3Kα)* |
| 1 | +++ | +++ | 14 | +++ | +++ |
| 2 | +++ | +++ | 15 | +++ | +++ |
| 3 | +++ | +++ | 16 | +++ | +++ |
| 4 | +++ | +++ | 17 | +++ | +++ |
| 5 | +++ | +++ | 18 | +++ | +++ |
| 6 | ++ | +++ | 19 | +++ | +++ |
| 7 | +++ | +++ | 20 | +++ | +++ |
| 8 | +++ | +++ | 21 | +++ | +++ |
| 9 | +++ | +++ | 22 | +++ | +++ |
| 10 | +++ | +++ | 23 | +++ | +++ |
| 11 | +++ | +++ | 24 | +++ | +++ |
| 12 | +++ | +++ | 25 | +++ | +++ |
| 13 | +++ | +++ | | | |

*+++ <1 μM
++ 1 μM-5 μM
+ >5 μM

The details of specific embodiments described in this invention are not to be construed as limitations. Various equivalents and modifications may be made without departing from the essence and scope of this invention, and it is understood that such equivalent embodiments are part of this invention.

1. A compound of formula (I): 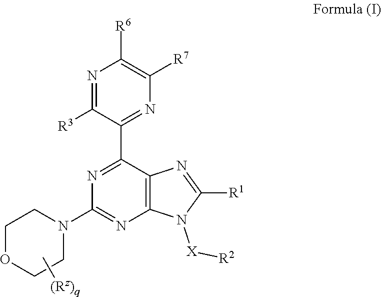
wherein:
R¹ is selected from the group consisting of: H, halogen and optionally substituted C₁-C₆ alkyl;
R² is selected from the group consisting of H, halogen, OH, NO₂, CN, NH₂, optionally substituted C₁-C₁₂alkyl, optionally substituted C₂-C₁₂alkenyl, optionally substituted C₂-C₁₂alkynyl, optionally substituted C₂-C₁₂heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, optionally substituted C₁-C₁₈ heteroaryl, optionally substituted C₁-C₁₂alkyloxy, optionally substituted C₂-C₁₂alkenyloxy, optionally substituted C₂-C₁₂alkynyloxy, optionally substituted C₂-C₁₀heteroalkyloxy, optionally substituted C₃-C₁₂cycloalkyloxy, optionally substituted C₃-C₁₂cycloalkenyloxy, optionally substituted C₂-C₁₂heterocycloalkyloxy, optionally substituted C₂-C₁₂ heterocycloalkenyloxy, optionally substituted C₆-C₁₈aryloxy, optionally substituted C₁-C₁ heteroaryloxy, optionally substituted C₁-C₁₂alkylamino, SR⁸, SO₃H, SO₂NR⁸R⁹, SO₂R⁸, SONR⁸R⁹, SOR⁸, COR⁸, COOH, COOR^{S}, CONR⁸R⁹, NR⁸COR⁹, NR⁸COOR⁹, NR⁸SO₂R⁹, NR⁸CONR⁸R⁹, NR⁸R⁹, and acyl;
R³ is selected from the group consisting of H, F, Cl, Br, OH, optionally substituted C₁-C₆ alkyl, OR⁸, OCOR⁸, CH₂OH, NH₂, NR⁸R⁹, NR⁸COR⁹, and NR⁸SO₂R⁹;
R⁶ is selected from the group consisting of H, OH, OR⁸, OP_{g}^{O}, OCOR⁸, CH₂OH, NH₂, NR⁸R⁹, NR⁸P_{g}^{N}, N(P_{g}^{N})₂, NR⁸COR⁹, and NR⁸SO₂R⁹;
R⁷ is selected from the group consisting of H, F, Cl, Br, OH, OR⁸, OCOR⁸, CH₂OH, NH₂, NR⁸R⁹, NR⁸COR⁹, and NR⁸SO₂R⁹;
each R⁸ and R⁹ is independently selected from the group consisting of H, optionally substituted C₁-C₁₂alkyl, optionally substituted C₂-C₁₂alkenyl, optionally substituted C₂-C₁₂alkynyl, optionally substituted C₂-C₁₀heteroalkyl, optionally substituted C₃-C₁₂cycloalkyl, optionally substituted C₃-C₁₂cycloalkenyl, optionally substituted C₂-C₁₂heterocycloalkyl, optionally substituted C₂-C₁₂heterocycloalkenyl, optionally substituted C₆-C₁₈aryl, and optionally substituted C₁-C₁₈heteroaryl, or
R⁸ and R⁹ when taken together with the atoms to which they are attached form an optionally substituted cyclic moiety;
P_{g}^{O} is a protecting group for oxygen;
each P_{g}^{N} is independently a protecting group for nitrogen;
each R^{z} is independently selected from the group consisting of C₁-C₆alkyl, halo-C₁-C₆ alkyl, hydroxyC₁-C₆alkyl, C₁-C₆alkyloxyC₁-C₆alkyl, cyanoC₁-C₆alkyl, aminoC₁-C₆alkyl, C₁-C₆alkylaminoC₁-C₆ alkyl, and di(C alkyl)amino C₁-C₆alkyl;
q is an integer selected from the group consisting of 0, 1, 2, 3, and 4;
X is a group of formula (CR¹⁰₂)ₘ;
each R¹⁰ is independently selected from the group consisting of: H and optionally substituted C₁-C₆ alkyl;
m is an integer selected from the group consisting of 0, 1, 2, 3 and 4;
or a pharmaceutically acceptable salt, N-oxide, or prodrug thereof.

2. A compound according to claim 1 wherein q is 0.

3. (canceled)

4. A compound according to claim 1 wherein R³ is selected from the group consisting of H, methoxy and methyl.

5. A compound according to claim 1 wherein R³ is H and R⁷ is H.

6. (canceled)

7. A compound according to claim 1 wherein m is selected from the group consisting of 0, 1, and 2.

8. (canceled)

9. (canceled)

10. A compound according to claim 1 wherein m is 1.

11. A compound according to claim 10 wherein one R¹⁰ is H and X is a group of the formula:

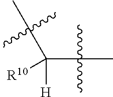

12. A compound according to claim 1 wherein the compound is a compound of the formula: 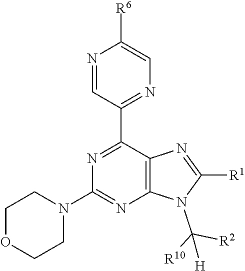
wherein R¹, R², R⁶ and R¹⁰ are as defined in claim 1.

13. (canceled)

14. A compound according to claim 12 wherein R¹⁰ is selected from the group consisting of H, methyl, ethyl, propyl, isopropyl, and butyl.

15. (canceled)

16. A compound according to claim 12 wherein R¹ is H.

17. (canceled)

18. A compound according to claim 12 wherein R⁶ is NH₂.

19. A compound according to claim 12 wherein R² is selected from the group consisting of H, cyano, optionally substituted C₁-C₁₂alkyl, optionally substituted C₂-C₁₂alkenyl, optionally substituted C₂-C₁₂heteroalkyl, optionally substituted C₃-C₁₂cycloalkyl, optionally substituted C₂-C₁₂heterocycloalkyl, optionally substituted C₆-C₁₈aryl, and optionally substituted C₁-C₁₈heteroaryl.

20-31. (canceled)

32. A compound according to claim 12 wherein the optional substituent is selected from the group consisting of: F, Br, Cl, ═O, ═S, —CN methyl, trifluoro-methyl, ethyl, 2,2,2-trifluoroethyl, isopropyl, propyl, 2-ethyl-propyl, 3,3-dimethyl-propyl, butyl, isobutyl, 3,3-dimethyl-butyl, 2-ethyl-butyl, pentyl, 2-methyl-pentyl, pent-4-enyl, hexyl, heptyl, octyl, phenyl, NH₂, —NO₂, phenoxy, hydroxy, methoxy, trifluoro-methoxy, ethoxy, and methylenedioxy.

33. A compound according to claim 1 selected from the group consisting of: 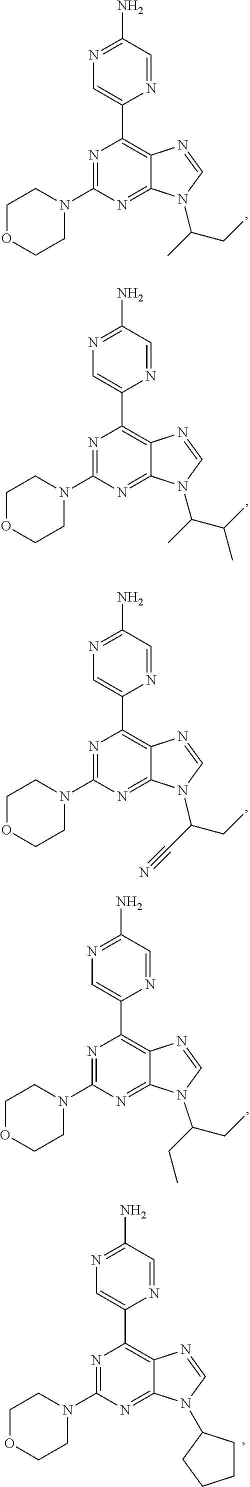 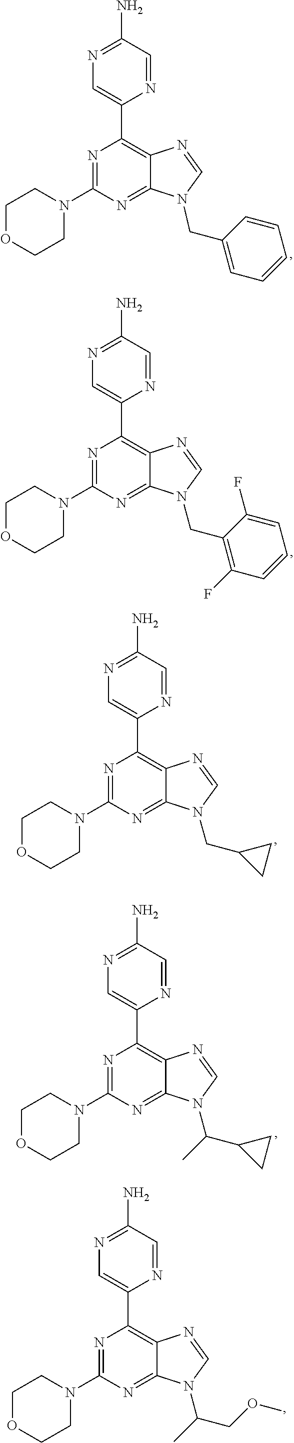 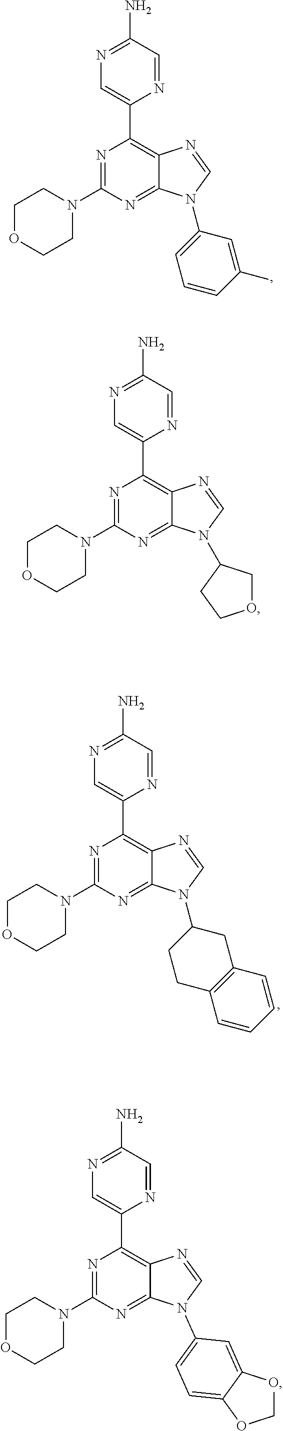 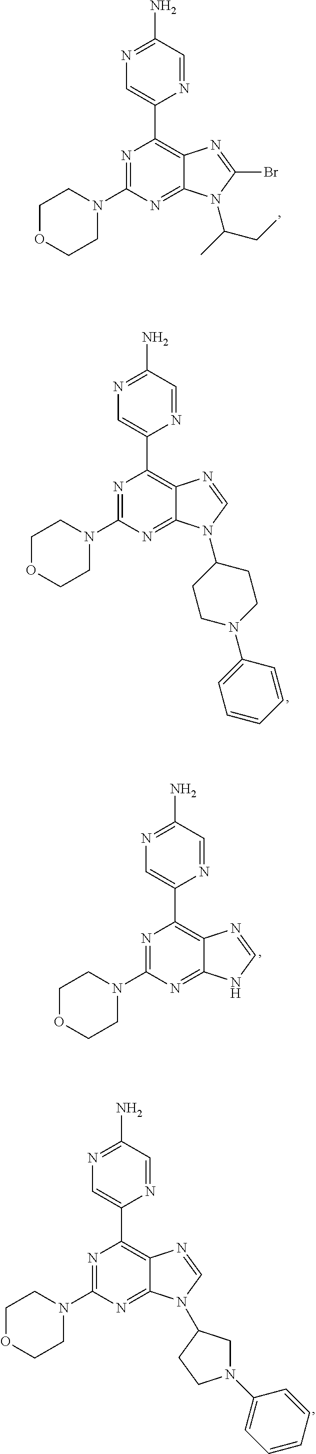 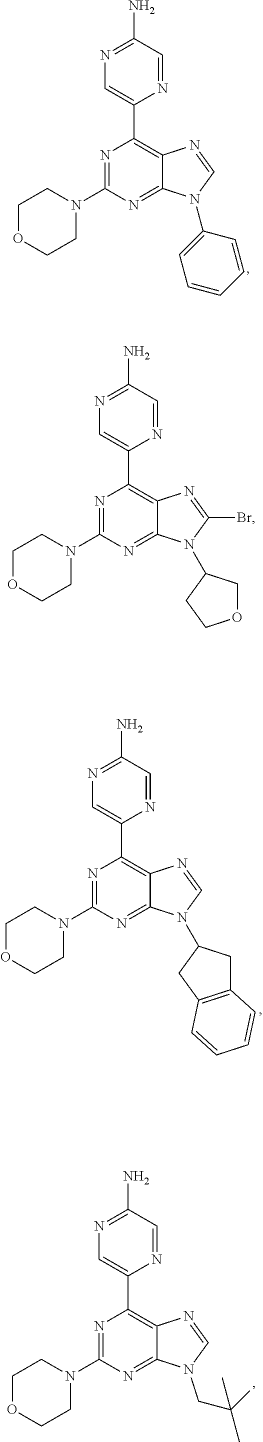 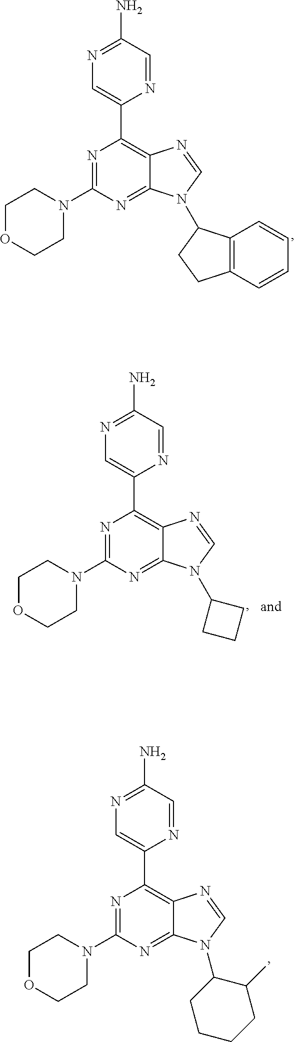
or a pharmaceutically acceptable salt or prodrug thereof.

34. A pharmaceutical composition including a compound according to claim 1 and a pharmaceutically acceptable diluent, excipient or carrier.

35. A method of inhibiting a protein kinase selected from the group consisting of a serine/threonine protein kinase or a fragment or a complex thereof or a functional equivalent thereof and a PI3 kinase or a fragment or a complex thereof or a functional equivalent thereof, the method including exposing the protein kinase or a fragment or complex thereof or a functional equivalent thereof and/or co-factor(s) thereof to an effective amount of a compound according to claim 1.

36. (canceled)

37. A method according to claim 35 wherein the serine/threonine protein kinase or a fragment or complex thereof is an mTOR protein kinase or a fragment thereof, or a complex thereof or a functional equivalent thereof.

38. (canceled)

39. A method according to claim 35 wherein the protein kinase is a PI3 kinase or a fragment thereof or a complex thereof or a functional equivalent thereof.

40-46. (canceled)

47. A method of treating or preventing a condition in a mammal in which inhibition of one or more protein kinase(s) selected from the group consisting of a serine/threonine protein kinase or a fragment or a complex thereof or a functional equivalent thereof and a PI3 kinase or a fragment or a complex thereof or a functional equivalent thereof, prevents, inhibits or ameliorates a pathology or a symptomology of the condition, the method including administration of a therapeutically effective amount of a compound according to claim 1.

48-52. (canceled)

53. A method according to claim 47 wherein the condition is cancer.

54. A method according to claim 53 wherein the cancer is selected from the group consisting of Hematologic cancer such as myeloproliferative disorders (idiopathic myelofibrosis, polycythemia vera, essential thrombocythemia, chronic myeloid leukemia), myeloid metaplasia, chronic myelomonocytic leukemia, acute lymphocytic leukemia, acute erythroblastic leukemia, Hodgkin's and Non Hodgkin's disease, B-cell lymphoma, acute T-cell leukemia, myelodysplastic syndromes, plasma cell disorder, hairy cell leukemia, kaposi's sarcoma, lymphoma; gynaecologic cancer such as breast carcinoma, ovarian cancer, cervical cancer, vaginal and vulva cancer, endometrial hyperplasia; gastrointestinal tract cancer such as colorectal carcinoma, polyps, liver cancer, gastric cancer, pancreatic cancer, gall bladder cancer; urinary tract cancer such as prostate cancer, kidney and renal cancer; urinary bladder cancer, urethral cancer, penile cancer; skin cancer such as melanoma; brain tumour such as glioblastoma, neuroblastoma, astrocytoma, ependynoma, brain-stem gliomas, medulloblastoma, menigiomas, astrocytoma, oligodendroglioma; head and neck cancer such as nasopharyngeal carcinoma, laryngeal carcinoma; respiratory tract cancer such as lung carcinoma (NSCLC and SCLC), mesothelioma; eye disease such as retinoblastoma; musculo-skeleton diseases such as osteosarcoma, musculoskeleletal neoplasm; Squamous cell carcinoma and fibroid tumour.

55-63. (canceled)

64. A method of prevention or treatment of a proliferative condition in a subject, the method including administration of a therapeutically effective amount of a compound according to claim 1 to the subject.

65. A method according to claim 64 wherein the condition is cancer.

66. A method according to claim 64 wherein the cancer is selected from the group consisting of Hematologic cancer such as myeloproliferative disorders (idiopathic myelofibrosis, polycythemia vera, essential thrombocythemia, chronic myeloid leukemia), myeloid metaplasia, chronic myelomonocytic leukemia, acute lymphocytic leukemia, acute erythroblastic leukemia, Hodgkin's and Non Hodgkin's disease, B-cell lymphoma, acute T-cell leukemia, myelodysplastic syndromes, plasma cell disorder, hairy cell leukemia, kaposi's sarcoma, lymphoma; gynaecologic cancer such as breast carcinoma, ovarian cancer, cervical cancer, vaginal and vulva cancer, endometrial hyperplasia; gastrointestinal tract cancer such as colorectal carcinoma, polyps, liver cancer, gastric cancer, pancreatic cancer, gall bladder cancer; urinary tract cancer such as prostate cancer, kidney and renal cancer; urinary bladder cancer, urethral cancer, penile cancer; skin cancer such as melanoma; brain tumour such as glioblastoma, neuroblastoma, astrocytoma, ependynoma, brain-stem gliomas, medulloblastoma, menigiomas, astrocytoma, oligodendroglioma; head and neck cancer such as nasopharyngeal carcinoma, laryngeal carcinoma; respiratory tract cancer such as lung carcinoma (NSCLC and SCLC), mesothelioma; eye disease such as retinoblastoma; musculo-skeleton diseases such as osteosarcoma, musculoskeleletal neoplasm; Squamous cell carcinoma and fibroid tumour.

67-70. (canceled)
